(19) United States
(12) Patent Application Publication
(10) Pub. No.: US 2005/0042641 A1
(43) Pub. Date: Feb. 24, 2005

(54) In vivo high throughput selection of RNAi probes

(75) Inventors: Vivek Mittal, Syosset, NY (US); Rajeev Kumar, Great Falls, MT (US)

Correspondence Address: DARBY & DARBY P.C., P. O. BOX 5257, NEW YORK, NY, 10150-5257, US

(73) Assignee: Cold Spring Harbor Laboratory, Cold Spring Harbor, NY (US)

(21) Appl. No.: 10/854,777

(22) Filed: May 25, 2004

Related U.S. Application Data
(60) Provisional application No. 60/473,809, filed on May 27, 2003.

(51) Int. Cl. C12Q 1/68

(52) U.S. Cl. 435/6

(57) ABSTRACT

In mammalian systems, RNA interference (RNAi)-based suppression of target gene expression may be activated by delivery of RNAi probes such as double stranded small interfering RNA (siRNA) molecules or short hairpin RNAs (shRNAs), where the RNAi probe sequence is homologous to the target gene. A reliable and quantitative method is provided for the rapid and efficient identification of RNAi probes that are most effective in providing RNAi-mediated suppression of target gene expression. This method may be used for high-throughput screens to identify effective RNAi probes.

This application claims priority to U.S. Ser. No. 60/473,809, filed on May 27, 2003. This prior application is incorporated herein by reference.

### FIELD OF THE INVENTION

In mammalian systems, RNA interference (RNAi)-based suppression of target gene expression may be activated by delivery of RNAi probes such as double stranded small interfering RNA (siRNA) molecules or short hairpin RNAs (shRNAs), where the RNAi probe sequence is homologous to the target gene. A reliable and quantitative method is provided for the rapid and efficient identification of RNAi probes that are most effective in providing RNAi-mediated suppression of target gene expression. This method may be used for high-throughput screens to identify effective RNAi probes.

### BACKGROUND OF THE INVENTION

RNA interference (RNAi) is a process of sequence-specific post-transcription gene silencing by which double-stranded RNA (dsRNA) homologous to a target locus can specifically inactivate gene function in plants, invertebrates and mammalian systems (Hammond, et al. Nat Genet. 2001;2:110-119; Sharp. Genes Dev 1999;13:139-141). This dsRNA induced gene silencing is mediated by 21- and 22-nucleotide double stranded small interfering RNAs (siRNAs) generated from longer dsRNAs by ribonuclease III cleavage (Bernstein, et al. Nature 2001;409:363-366; and Elbashir, et al. Genes Dev 2001;15:188-200). RNAi-mediated gene silencing is thought to occur via sequence-specific mRNA degradation, where sequence specificity is determined by the interaction of an siRNA with its complementary sequence within a target MRNA (see, e.g., Tuschl, Chem Biochem 2001;2:239-245).

For mammalian systems, RNAi may be activated by introduction of either siRNAs (Elbashir, et al. Nature 2001;411:494-498) or short hairpin RNAs (shRNAs) bearing a fold back stem-loop structure (Paddison, et al. Genes Dev 2002;16:948-958; Sui, et al. Proc Natl Acad Sci USA 2002;99:5515-5520; Brummelkamp, et al. Science 2002;296:550-553; and Paul, et al. Nat Biotechnol 2002;20:505-508).

Although general guidelines for designing siRNA oligonucleotides are available (Elbashir, et al. Methods 2002;26: 199-213), the majority of siRNAs or shRNAs designed against a gene are not effective for silencing gene expression in mammals (Bernstein, et al. Nature 2001;409:363-366; Elbashir, et al. supra; Holen, et al. Nucleic Acids Res 2002;30:1757-1766; Lee, et al. Nat Biotechnol 2002;20:500-505; Yu, et al. Proc Natl Acad Sci USA 2002;99:6047-6052; and Kapadia, et al. Proc Natl Acad Sci USA. 2003;3:2014-2018). Roughly 1 in 5 of the siRNAs/shRNAs selected for targeting a region of a gene provide efficient gene silencing (Kapadia, et al. supra; McManus, et al. RNA 2002;8:842-850; and our observations). Although empirical data elucidating the cause of failures associated with a majority of siRNAs are unavailable, various factors including, instability of siRNA probe in vivo, inability to interact with components of the RNAi machinery, or inaccessibility of the target mRNA pertaining to local secondary structural constraints may be responsible. Analysis of nucleotide sequences, melting temperatures, and secondary structures has not yet revealed any obvious differences between effective and non-effective siRNA (Hohjoh, FEBS Lett. 2002;521:195-199).

Moreover, empirical approaches that provide for reliable and efficacious identification of siRNA or shRNA probes have not yet been developed. An RNAseH susceptibility assay for siRNA/target duplex has been proposed (Lee, et al. supra). In this assay the degree of RNaseH sensitivity reflects the accessibility of the chosen site in the target gene. However, this approach is time-consuming and its general applicability has not been established. A “shotgun” approach has also been proposed (Yang, et al. Proc Natl Acad Sci USA 2002;99:9942-9947; Calegari, et al. Proc Natl Acad Sci U S A. 2002;99: 14236-14240). In this approach, a mixture of siRNA produced by RNAseIII mediated hydrolysis of long double-stranded RNA is used as the RNAi probe. However, this method does not allow one to distinguish specific versus non-specific effects on gene silencing as a consequence of the presence of many cleavage products in the mixture.

Thus, although RNAi has recently emerged as a powerful genetic tool to suppress gene expression and/or analyze gene function in mammalian cells, the power of this method has been limited by the uncertainty in predicting the efficacy of a particular siRNA or shRNA in silencing a gene, and by the distinct lack of a siRNA/shRNA selection algorithm or method. This uncertainty in siRNA/shRNA design has imposed serious limitations not only for small-scale, but also for high throughput RNAi analysis initiatives in mammalian systems.

We have developed a reliable and quantitative procedure for rapid and efficient identification of effective RNAi probes (e.g., siRNAs and/or shRNAs) for inhibition of target gene expression. Effective RNAi probes are identified based on their ability to inactivate cognate sequences in an ectopically expressed target-reporter fusion transcript. The effect of an RNAi probe may be monitored quantitatively. By examining a variety of genes with diverse biological functions, we have shown a strong correlation in the ability of siRNA or shRNA probes to suppress expression of ectopically expressed target-reporter fusions, with their ability to suppress expression of endogenous target gene counterparts. Furthermore, using microarray based cell transfections we demonstrate that this approach can be tailored to high throughput screens for identifying effective siRNA or shRNA probes in mammalian systems. The ability to successfully identify effective RNAi probes for silencing any gene will have significant implications not only in basic research, but also in RNAi based therapeutics (Agami. Curr Opin Chem Biol. 2002;6:829-834; Cottrell, et al. Trends Microbiol. 2003;11:37-43; and Shi, Trends Gene. 2003;19:9-12) and generation of genetically modified animal models (Carmell, et al. Nat Struct Biol. 2003;10:91-92; Hasuwa, et al. FEBS Lett. 2002;532:227-230; and Kim, et al. Biochem Biophys Res Commun. 2002;296: 1372-1377).

### SUMMARY OF THE INVENTION

The present invention is directed to a method of determining whether an RNAi probe can inhibit expression of a target gene, which method comprises detecting expression of (i) a target-reporter fusion construct in a first cell transfected with a candidate RNAi molecule and the target-reporter fusion construct, wherein the target-reporter fusion construct comprises a reporter gene fused to the target nucleic acid, and (ii) the target-reporter fusion construct in a second cell transfected with the target-reporter fusion construct, wherein the candidate RNAi molecule inhibits expression of the target nucleic acid if the level of target-reporter fusion expression in the first cell is decreased as compared to the level of expression in the second cell. In an exemplified embodiment of the method, the reporter is a fluorescent reporter and the detecting is done by measuring fluorescence intensity. In another exemplified embodiment, the reporter is an enzymatic reporter. In one embodiment of the method, the target-reporter fusion construct comprises a reporter gene-encoding sequence fused to the 5′ end of the target nucleic acid sequence. An in alternate embodiment, the target-reporter fusion construct comprises a reporter gene-encoding sequence fused to the 3′ end of the target nucleic acid sequence. In exemplified embodiments, the first and second cells are mammalian cells.

The invention is further directed to a high-throughput method of screening for candidate RNAi molecules that inhibit expression of a target nucleic acid, which method comprises (a) arraying candidate RNAi molecules and a target-reporter fusion construct onto a surface, wherein the target-reporter fusion construct comprises a reporter gene fused to the target nucleic acid, and each candidate RNAi molecule is localized to a spatially distinct spot on the surface; (b) incubating the arrayed surface with cells under appropriate conditions for entry of nucleic acid molecules, wherein this incubation results in clusters of transfected cells; and (c) detecting expression of the target-reporter fusion in the clusters of transfected cells, wherein a candidate RNAi molecule inhibits expression of the target nucleic acid if the level of target-reporter fusion expression in the cluster of cells into which the candidate RNAi molecule was transfected is decreased as compared to the level of expression in other clusters of cells. In an exemplified embodiment, a protein carrier is also arrayed onto the surface, and the surface is a glass slide. In an exemplified embodiment the reporter is a fluorescent reporter, and the detecting is done by measuring fluorescence intensity.

The invention is also directed to a high-throughput method of screening for candidate RNAi molecules that inhibit expression of a target nucleic acid, which method comprises (a) depositing a nucleic acid-containing mixture onto a surface in discrete, defined locations, wherein the nucleic acid-containing mixture comprises a target-reporter fusion construct comprising a reporter gene fused to the target nucleic acid, a candidate RNAi molecule, and a carrier protein and allowing the nucleic acid-containing mixture to dry on the surface, thereby producing a surface having the nucleic acid-containing mixture affixed thereon in discrete, defined locations, (b) plating eukaryotic cells onto the surface in sufficient density and under appropriate conditions for entry of nucleic acid in the nucleic acid-containing mixture into the eukaryotic cells, whereby nucleic acid in the nucleic acid-containing mixture is introduced into the eukaryotic cells, resulting in clusters of transfected cells; and (c) detecting expression of the target-reporter fusion in the clusters of transfected cells, wherein a candidate RNAi molecule inhibits expression of the target nucleic acid if the level of target-reporter fusion expression in the cluster of cells into which the RNAi probe was transfected is decreased as compared to the level of expression in other clusters of transfected cells. In an exemplified embodiment, a protein carrier is also arrayed onto the surface, and the surface is a glass slide. In an exemplified embodiment the reporter is a fluorescent reporter, and the detecting is done by measuring fluorescence intensity.

### DESCRIPTION OF THE DRAWINGS

FIG. 1 depicts the strategy and experimental verification of a screen for effective RNAi probes using a target-reporter fusion. A panel of siRNAs or shRNAs against a target gene (▴) is screened using an expression construct wherein a reporter gene is fused at the 3′ end of a target gene, or the 5′ end of a target gene. Efficacy of siRNA mediated target gene silencing is measured by quantitation of reporter gene expression.

FIG. 2A depicts the pSHAG-1 vector used to assemble the shRNA expression constructs. FIG. 2B depicts the sequence of the human U6 promoter (SEQ ID NO: 1) contained in the pSHAG-1 vector (“U6 pro” in FIG. 2A) and the pSHAG-Ff1 vector (“U6 pro” in FIG. 3). The site of transcription intiation is indicated (“+1”).

FIG. 3 depicts the pShag-Ff1 expression construct used for expression of the non-specific shRNA control (NON-SP shRNA). The vector contains a Firefly luciferase-specific sequence inserted in to the EcoRV site (in bold) of the vector. The site of transcription intiation is indicated (“+1”).

FIG. 4 depicts target gene-specific siRNA mediated target gene and reporter gene silencing. Normalized relative amount of Renilla and Firefly luciferase (REN LUC/FF LUC) (n=3) is plotted as a function of treatment with increasing concentrations of non-specific siRNA (□) or EGFP-specific siRNA (▴).

FIGS. 5A, B, and C show the correlation between siRNA and shRNA screening results and suppression of endogenous MyoD expression. (A) The normalized fluorescence intensity ratio (Normalized GFP/RFP) of target (MyoD-EGFP) to the internal control (RFP) was quantitated for each MyoD-specific siRNA and a non-specific siRNA (NON-SP) by examining protein lysates from transfected cells. (B) Murine C2C12 cells transfected with MyoD-specific siRNA or a non-specific siRNA (NON-SP) were subjected to Western blot analysis for MyoD and α-tubulin proteins. (C) Murine C2C12 cells transfected with MyoD-specific shRNAs or a non-specific siRNA (NON-SP) were subjected to Western blot analysis for MyoD and a-tubulin proteins.

FIG. 6 shows the correlation between siRNA screening results and suppression of endogenous Lamin A/C expression. HeLa cells transfected with Lamin A/C-specific siRNA or non-specific siRNA (NON-SP) were subjected to Western blot analysis for Lamin A/C and a-tubulin proteins.

FIG. 7 depicts a laser scan of EGFP and RFP fluorescence images of HeLa cell clusters on microarray. The cell clusters have been transfected with target gene expression constructs (pEGFP-N2 or MyoD-EGFP), pDsRed2-N1, and varying concentrations of EGFP-specific or non-specific siRNAs using a microarray based cell transfection method.

FIG. 8 depicts the dose dependent effect of EGFP-specific siRNA in suppression of EGFP expression as quantitated by normalized mean intensities of fluorescence (Mean EGFP/RFP). Mean EGFP/RFP (n=4) is plotted as a function of treatment with increasing concentrations (ng) of EGFP-SP siRNA (♦).

FIG. 9A and B depict the results of microarray-based screens for RNAi probes that are effective against the MyoD gene. Mean intensities of fluorescence (EGFP/RFP) were log transformed, normalized (n=4), and plotted in a graph on the Y-axis versus individual RNAi probes on the X-axis. RNAi probes within 1 standard deviation (1 s.d.) from the mean value were considered non-effective; and those outside 1 standard deviation (1 s.d.) were considered effective. (A) A screen for shRNA effective against the MyoD gene identified shRNA 708 as most effective. (B) A screen for siRNA effective against the MyoD gene identified siRNA 25 as most effective.

### DETAILED DESCRIPTION

The invention provides a reliable and quantitative approach for the rapid and efficient identification of an effective RNAi probe against any gene, and for selecting the best RNAi probe from among a group of RNAi candidates. This method may be used for high-throughput screens (e.g., based on microarray cell transfections) of RNAi probes. A major strength of this method is its ability to identify the most robust RNAi probe for a target gene in an mammalian system within 24 hours. This method, therefore, has great potential for identifying effective RNAi probes.

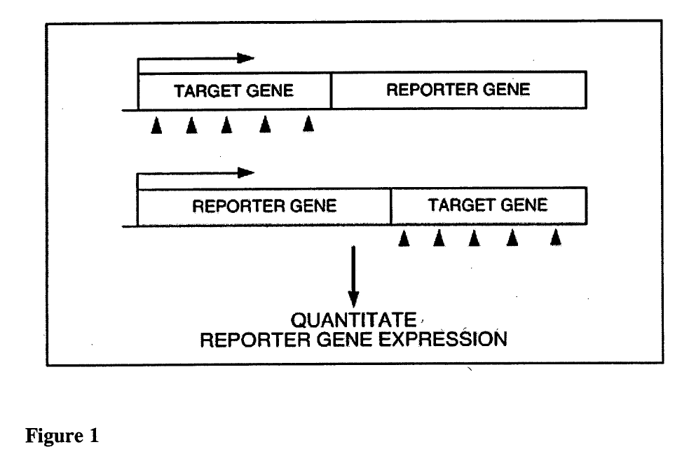

The method is based upon introduction into a target cell of both an RNAi probe and a cognate target-reporter fusion expression construct, where expression of the target-reporter fusion may be easily quantitated based upon the reporter. The target-reporter fusions are encoded by expression constructs wherein a sequence encoding the target gene of interest is fused to a reporter gene. The reporter gene sequences may be fused to the 5′ end or the 3′ end of the target gene sequences (see FIG. 1). Such fusion may result, for example, in the translation of a fusion protein in which the reporter protein is fused N-terminal or C-terminal of the protein encoded by the target gene. Thus, the method allows for substantial flexibility in the construction of target-reporter fusions.

The efficacy of an RNAi probe is determined by its ability to reduce the expression of the target-reporter fusion. If the RNAi probe effectively targets and inactivates expression of its target gene a marked reduction in reporter expression (e.g., EGFP/RFP fluorescence or Luciferase enzymatic activity) is observed; and conversely if it fails to efficiently target its target gene a significant change in reporter expression is not observed. Both of these activities are subject to quantitation.

The ability of an RNAi probe to suppress target-reporter fusion expression (as quantitated by reporter expression) specifically correlates with the ability of the identified RNAi probe to effectively suppress expression of the cognate endogenous gene. Thus, this method is particularly advantageous in identifying effective RNAi probes for target genes for which probes to monitor suppression of endogenous gene expression (e.g., antibodies, RT-PCR primers, or Northern blot hybridization probes) are either unavailable or unreliable.

In addition to identifying the most effective RNAi probe for a target gene, this quantitative method allows for the identification of RNAi probes that provide partial suppression of target gene expression. These RNAi probes may also be useful, for example, for applications where lethality associated with complete suppression of critical genes is of concern, or where partial down regulation of gene expression results in a discrete phenotype. For example, shRNAs showing varying levels of p53 suppression generated distinct tumor phenotypes in vivo (Hemann, et al. Nat Genet. 2003;33:396-400).

As used herein, the term “RNA interference probe” or “RNAi probe” refers to synthetic or natural ribonucleic acid species, or derivatives thereof, which are intended to induce RNA interference (RNAi)-mediated suppression of target gene expression when introduced into a target cell. “RNAi probes” include small interfering RNAs (siRNAs) and short hairpin RNAs (shRNAs). These RNAi probes comprise sequences that are specific to a segment of the sequence of the target gene. The term “RNAi probes” also encompasses the expression constructs used for in vivo synthesis of siRNAs and shRNAs. A ribonucleic acid molecule can be tested for its suitability as an RNAi probe using the assay of the invention, described in greater detail below. Such a tested ribonucleic acid molecule may be termed an “RNAi candidate” or a “candidate RNAi molecule”. The present invention provides a rapid and convenient method to validate RNAi candidate molecules.

As used herein, the term “target gene” or “target nucleic acid” refers to any nucleic acid sequence capable of transcription into RNA, or capable of affecting transcription of a nucleic acid sequence into RNA. Target genes include, for example; genomic or mitochondrial DNA encoding mRNAs, tRNAs and rRNAs; genomically integrated transgenes; extrachromosmal DNA present in a target cell; and the DNA or RNA of a pathogen residing in the target cell. Such extrachromosomal elements include plasmids, cosmids, yeast artificial chromosomes, and the like. Such pathogens include transposable elements; RNA and DNA viruses, including retroviruses; protozoan parasites; fungi; bacteria; and the like. The RNAi probes may be specific to transcribed or untranscribed portions of the target gene. Preferably, the RNAi probes are complementary to transcribed portions of the target gene. Transcribed portions of the target gene to which RNAi probes may be complementary include introns, exons, 5′ untranslated sequences, and 3′ untranslated sequences. Non-coding region of the target gene to which RNAi probes may be complementary include 5′ untranslated regions, introns, and a 3′ untranslated regions. In particularly preferred embodiments, RNAi probes are complementary to exonic portions of the target gene.

As used herein, the term “target cell” refers to any cell into which an RNAi probe is introduced with the intent of inducing RNAi-mediated suppression of target gene expression. Target cells include, but are not limited to, bacteria, fungi, protozoan parasites, yeast, plant cells, and cells of invertebrate and vertebrate organisms. More particularly, target cells are mammalian cells, e.g., murine or human cells. Exemplified mammalian cells are mammalian cell lines cultured in vitro, particularly human HeLa cells and murine C2C12 cells.

As used herein, the term “reporter gene” encompasses any gene whose expressed product confers an assayable phenotype upon a cell expressing such a reporter gene. The expressed product of a reporter gene may be a transcribed RNA or a translated protein. Usually, the expressed product of a reporter gene is a protein, such as a fluorescent or enzymatic reporter. Exemplary fluorescent reporters include, but are not limited to, cyan fluorescent protein (CFP, also known as blue fluorescent protein), yellow fluorescent protein (YFP), green fluorescent protein (EGFP), and red fluorescent protein (RFP). Enzymatic reporters include, but are not limited to, alkaline phosphatase (AP), horseradish peroxidase (HRP), beta-galactosidase (LacZ), beta-glucoronidase (GUS), nopaline synthase (NOS), octapine synthase (OCS), acetohydroxyacid synthase (AHAS), chloramphenicol transferase (CAT), and luciferase (LUC) proteins. Specific luciferase reporters include Renilla luciferase and firefly luciferase proteins. In alternative embodiments, the reporter gene may encode a protein sequence conveniently detected by immunoassay methods, such as Western blotting, immunohistochemistry, ELISA, and/or immunoprecipitation. Exemplary embodiments of such protein sequences include His-tags, immunoglobulin domains, myc tags, poly-glycine tags, FLAG tags, HA-tags, and the like.

The recombinant DNA methods employed in practicing the present invention are standard procedures, well-known to those skilled in the art (as described, for example, in “Molecular Cloning: A Laboratory Manual.” 2^{nd} Edition. Sambrook, et al. Cold Spring Harbor Laboratory:1989, “A Practical Guide to Molecular Cloning” Perbal:1984, and “Current Protocols in Molecular Biology” Ausubel, et al., eds. John Wiley & Sons: 1989). These standard molecular biology techniques can be used to prepare the expression constructs of the invention.

### RNAI Candidates and Probes

### Small Interfering RNAs (siRNAs)

The siRNAs to be screened in accordance with the present invention are short double stranded nucleic acid duplexes comprising annealed complementary single stranded nucleic acid molecules. In preferred embodiments, the siRNAs to be screened in accordance with the present invention are short double stranded RNAs comprising annealed complementary single strand RNAs. However, the invention also encompasses embodiments in which the siRNAs comprise an annealed RNA:DNA duplex, wherein the sense strand of the duplex is a DNA molecule and the antisense strand of the duplex is a RNA molecule.

Preferably, each single stranded nucleic acid molecule of the siRNA duplex is of from about 21 nucleotides to about 27 nucleotides in length. In preferred embodiments, duplexed siRNAs have a 2 or 3 nucleotide 3′ overhang on each strand of the duplex. In preferred embodiments, siRNAs have 5′-phosphate and 3′-hydroxyl groups.

According to the present invention, siRNAs may be introduced to a target cell as an annealed duplex siRNA, or as single stranded sense and anti-sense nucleic acid sequences that once within the target cell anneal to form the siRNA duplex. Alternatively, the sense and anti-sense strands of the siRNA may be encoded on an expression construct that is introduced to the target cell. Upon expression within the target cell, the transcribed sense and antisense strands may anneal to reconstitute the siRNA.

### Short Hairpin RNAs (shRNAs)

The shRNAs to be screened in accordance with the present invention comprise a single stranded “loop” region connecting complementary inverted repeat sequences that anneal to form a double stranded “stem” region. Structural considerations for shRNA design are discussed, for example, in McManus, et al. RNA 2002;8:842-850. In certain embodiments the shRNA may be a portion of a larger RNA molecule, e.g., as part of a larger RNA that also contains U6 RNA sequences (Paul, et al. Nature Biotech 2002;20:505-508).

In preferred embodiments the loop of the shRNA is from about 0 to about 9 nucleotides in length. In preferred embodiments the double stranded stem of the shRNA is from about 19 to about 33 base pairs in length. In preferred embodiments, the 3′ end of the shRNA stem has a 3′ overhang. In particularly preferred embodiments, the 3′ overhang of the shRNA stem is from 1 to about 4 nucleotides in length. In preferred embodiments, shRNAs have 5′-phosphate and 3′-hydroxyl groups.

### Chemical Synthesis of RNAi Candidates and Probes

RNA molecules may be chemically synthesized, for example using appropriately protected ribonucleoside phosphoramidites and a conventional DNA/RNA synthesizer. Suppliers of RNA synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). For example, single-stranded gene-specific RNA oligomers may be synthesized using 2′-O-(tri-isopropyl) silyloxymethyl chemistry by Xeragon AG (Zurich, Switzerland). Alternatively, RNA oligomers may be synthesized using Expedite RNA phosphoramidites and thymidine phosphoramidite (Proligo). RNAs produced by such methodologies tend to be highly pure and to anneal efficiently to form siRNA duplexes or shRNA hairpin stem-loop structures.

Following chemical synthesis, single stranded RNA molecules are deprotected, annealed to form siRNAs or shRNAs, and purified (e.g., by gel electrophoresis or High Pressure Liquid Chromatography). For example, siRNAs may be generated by annealing sense and antisense single strand RNA (ssRNA) oligomers. Similarly, shRNAs may be generated by annealing of complementary sequences within a single ssRNA molecule to form a hairpin stem-loop structure. The integrity and the dsRNA character of the annealed RNAs may be confirmed by gel electrophoresis and quantified by spectroscopy (using the standard conversion, wherein 1 unit of Optical Density at 260 nm=40 ug of duplex RNA/ml).

Most conveniently, siRNAs may be obtained from commercial RNA oligomer synthesis suppliers, which sell RNA-synthesis products of different quality and cost. For example, commercial suppliers of siRNAs include Dharmacon, Xeragon Inc. (now a QIAGEN company), Proligo, and Ambion.

### In vitro Enzymatic Synthesis of RNAi Candidates and Probes

Standard procedures may used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been described (Donze and Picard. Nucleic Acids Res. 2002;30:e46; and Yu, et al. Proc. Natl. Acad. Sci. USA 2002;99:6047-6052). Similarly, an efficient in vitro protocol for preparation of shRNAs using T7 RNA polymerase has been described (Yu, et al. Proc. Natl. Acad. Sci. USA 2002;99:6047-6052).

For example, sense and antisense RNA oligonucleotides for siRNA preparation may be transcribed from a single DNA template that contains a T7 promoter in the sense and an SP6 promoter in the antisense direction. Alternatively, sense and antisense RNAs may be transcribed from two different DNA templates containing a single T7 or SP6 promoter sequence. The sense and antisense transcripts may be synthesized in two independent reactions or simultaneously in a single reaction. Similarly, a ssRNA may be synthesized from a DNA template encoding a shRNA. The transcribed ssRNA oligomers are then annealed and purified. siRNAs may be generated by annealing sense and antisense ssRNA oligomers. Similarly, shRNAs may be generated by annealing of complementary sequences within a single ssRNA molecule to form a hairpin stem-loop structure. The integrity and the dsRNA character of the annealed RNAs may be confirmed by gel electrophoresis and quantified by spectroscopy (using the standard conversion, wherein 1 unit of Optical Density at 260 nm=40 ug of duplex RNA/ml).

### In vivo Synthesis of RNAi Candidates and Probes within Target Cells

RNAi probes may be formed within the target cell by transcription of RNA from an expression construct introduced into the target cell. For example, a protocol and expression construct for in vivo expression of siRNAs is described in Yu, et al. supra. Similarly, protocols and expression constructs for in vivo expression of shRNAs have been described (Brummelkamp, et al. Science 2002;296:550-553; Sui, et al. Proc. Natl. Acad. Sci USA 2002;99:5515-5520; Yu, et al. supra; McManus, et al. RNA 2002;8:842-850; and Paul, et al. Nature Biotech 2002;20:505-508.

For example, an siRNA may be reconstituted in a target cell by use of an siRNA expression construct that upon transcription within the target cell produces the sense and antisense strands of the siRNA. These complementary sense and antisense RNAs then anneal to reconstitute the siRNA within the target cell. In one embodiment, the sense and antisense strands are encoded by a single sequence of the expression vector flanked by two promoters of opposite transcriptional orientation, thereby driving transcription of the alternate strands of the sequence. In another embodiment, the sense and antisense strands are encoded by independent sequences within a single expression vector, where each independent sequence is operably linked to a promoter to drive transcription. In yet another embodiment, the sense and antisense strands are encoded by independent sequences on two independent expression constructs, where each independent sequence is operably linked to a promoter to drive transcription.

Similarly, shRNAs may be generated in vivo by transcription of a single stranded RNA from an expression construct within the target cell. The complementary sequences of the inverted repeat within the ssRNA then anneal to yield the stem-loop structure of the shRNA.

Expression construct-encoded RNAi probes have distinct advantages over their chemically synthesized or in vitro transcribed counterparts. They are cost effective and provide a stable and continuous expression of RNAi probe that is useful for analysis of phenotypes that develop over extended periods of time.

The expression constructs for in vivo production of RNAi probes comprise RNAi probe encoding sequences operably linked to elements necessary for the proper transcription of the RNAi probe encoding sequence(s), including promoter elements and transcription termination signals. Preferred promoters for use in such expression constructs include the polymerase-III HI-RNA promoter (see, e.g., Brummelkamp, et al. supra) and the U6 polymerase-III promoter (see, e.g., Sui, et al. supra; Paul, et al. supra; and Yu, et al. supra).

The RNAi probe expression constructs may further comprise vector sequences that facilitate the cloning and propagation of the expression constructs. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest. Prolonged expression of the encoded RNAi probe in in vitro cell culture may be achieved by the use of vectors sequences that allow for autonomous replication of an extrachromosomal construct in mammalian host cells (e.g., EBNA-1 and oriP from the Epstein-Barr virus).

### Sequence Composition of RNAi Candidates and Probes

The RNAi candidates to be screened according to the present invention are specific to a portion of the chosen target gene. The RNAi candidates may be specific to transcribed or untranscribed portions of the target gene. In preferred embodiments, the RNAi probes are complementary to transcribed portions of the target gene. Transcribed portions of the target gene to which RNAi probes may be complementary include introns, exons, 5′ untranslated sequences, and 3′ untranslated sequences. In more preferred embodiments, RNAi probes are complementary to exonic portions of the target gene. Where multiple transcripts are produced from the same target gene (e.g., as from alternative splicing), RNAi probes are specific to a particular transcript if directed to a region of the transcript that is not contained within other transcripts produced from the target gene. For example, in the case of a target gene subject to alternative splicing, RNAi probes may be specific to an exon only present in certain of the transcripts. In this case, the RNAi pathway will suppress expression of transcripts containing that targeted exon, while allowing the other transcripts of the target gene (which do not contain the exon) to be expressed.

The RNAi candidates to be screened according to the invention preferably contain nucleotide sequences that are identical to a portion of the chosen target gene. However, RNA sequences with insertions, deletions, and single point mutations relative to the target sequence have also been found to be effective for RNAi mediated inhibition of target gene expression (see, e.g., U.S. Pat. No. 6,506,559). Therefore, 100% sequence identity between the RNAi probe and the target gene is not required to practice the invention. As such, RNAi candidates with insertions, deletions, and/or single point mutations relative to the target sequence may also be screened according to the present invention. Notably, in this respect, the current method provides the ability to determine rapidly and efficiently which sequence alterations are tolerated by the RNAi pathway.

The degree of sequence identity between an RNAi probe and its target gene may be determined by sequence comparison and alignment algorithms known in the art (see, for example, Gribskov and Devereux Sequence Analysis Primer (Stockton Press: 1991) and references cited therein). The percent similarity between the nucleotide sequences may be determined, for example, using the Smith-Waterman algorithm as implemented in the BESTFIT software program using default parameters. Greater than 90% sequence identity between the RNAi probe and the portion of the target gene corresponding to the RNAi probe is preferred.

### Modifications to RNAi Candidates and Probes

The RNA of RNAi probes may include one or more modifications, either to the phosphate-sugar backbone or to the nucleoside. For example, the phosphodiester linkages of natural RNA may be modified to include at least one heteroatom, such as nitrogen or sulfur. In this case, for example, the phosphodiester linkage may be replaced by a phosphothioester linkage. Similarly, bases may be modified to block the activity of adenosine deaminase. Where the RNAi candidate or probe is produced synthetically, or by in vitro transcription, a modified ribonucleoside may be introduced during synthesis or transcription. For example, incorporation of 2′-aminouridine, 2′-deoxythymidine, or 5′-iodouridine into the sense strand of an RNAi probe is tolerated by the RNAi pathway, whereas the same substitutions on the antisense strand of the RNAi is not (Parrish, et al. Mol Cell 2000;6:1077-87). Also, if a siRNA has a 2 or 3 nucleotide 3′ overhang on each strand of the duplex, substitution of 2′-deoxythymidine for uridine in the overhangs is tolerated by the RNAi pathway. The present invention provides a rapid and efficient system and method for introducing systematic variations into RNAi probes to create RNAi candidates with desirable chemical properties, e.g., a more stable phosphothioester linkage.

### Target-Reporter Fusions

The recombinant DNA methods employed in practicing the present invention are standard procedures, well-known to those skilled in the art (as described, for example, in “Molecular Cloning: A Laboratory Manual.” 2nd Edition. Sambrook, et al. Cold Spring Harbor Laboratory: 1989, “A Practical Guide to Molecular Cloning” Perbal: 1984, and “Current Protocols in Molecular Biology” Ausubel, et al., eds. John Wiley & Sons: 1989). These standard molecular biology techniques can be used to prepare the expression constructs of the invention.

For the screening method of the present invention a nucleic acid sequence encoding the selected target gene is fused to a nucleic acid sequence encoding the chosen reporter gene. Such linked nucleic acid sequences are referred to as “target-reporter fusions”. As used herein the term “target-reporter fusions” encompasses fusion sequences encoding an transcript that is not translated, as well as those encoding a transcript that is translated to produce a polypeptide.

In embodiments where the assayable phenotype of the reporter gene is based upon the presence of the reporter gene transcript, the two sequences are linked so as to maintain the proper transcriptional orientation for each sequence. Note that in this case, it is not strictly necessary to maintain the translational frame of either sequence. In one embodiment, the reporter gene sequences are linked to the 3′ of the target gene sequences. In another embodiment, the target gene sequences are linked to the 3′ end of the reporter gene sequences.

In embodiments where the assayable phenotype of the reporter gene is based upon the presence of a protein encoded by the reporter gene sequence, the two sequences are linked so as to maintain the proper transcriptional orientation for each sequence, and to maintain proper translation initiation and translational frame of the reporter gene sequence. Note that in this case, it is not strictly necessary to maintain the normal translational frame of the target gene sequence. For example, in one embodiment the target gene sequences may be linked to the 3′ end of sequences encoding the reporter protein. In this case, translation initiation sequences are located at the 5′ end of the fusion transcript to direct proper translation of the reporter protein: however it is not strictly necessary to maintain the translational frame of the downstrean target gene sequences. In another embodiment, sequences encoding the reporter protein are linked to the 3′ end of the target gene sequences. In this case, proper translation of the reporter protein may be provided by any of several mechanisms. For example, the two sequences (target and reporter) may be fused so as to encode a single fusion protein, where the translational frame is maintained across the fusion protein and translation initiation signals are provided at the 5′ end of the fusion transcript. In another embodiment, the two sequences may be fused such that the the target gene sequences are not preceded by any translation initiation sequences, while the reporter protein encoding sequences are. In this case, the target gene sequences will not be translated, but the reporter protein sequences will be translated in the appropriate frame. In yet another embodiment, both the target gene sequences and the reporter protein sequences are preceded by translation initiation sequences and independent translation of each polypeptide is provided by inclusion of an Internal Ribosomal Entry Site (IRES) element between the target gene sequences and the reporter protein sequences.

The nucleic acid sequence encoding the target gene may be a partial or complete sequence of the target gene. For example, in one embodiment the complete genomic DNA sequence of a target gene is used, while in another embodiment full length cDNA sequence is used. In yet another embodiment a partial sequence representing the sequence of a single exon of a multiple exon target gene is used. In another embodiment the sequence of a target gene promoter element may be used. The number of different RNAi candidates that may be screened using a given expression construct is directly proportional to the length of the target gene encoding sequence (i.e., the longer the target gene sequence, the greater number of candidates that may be screened).

The nucleic acid sequence encoding the reporter gene must be of sufficient length to confer the chosen assayable phenotype upon a cell expressing the reporter gene sequence. For example, where the reporter is to be detected based upon fluorescence from a green fluorescence protein, the sequence to be used must at minimum encode a translated polypeptide that fluoresces. In another example, where the reporter is to be detected based upon an immunoassay specific to a particular epitope tag, the sequence to be used must at minimum encode a translated polypeptide containing the specific epitope detected by the immunoassay.

These target-reporter fusion sequences are inserted into expression constructs for use in the screening method of the invention. In embodiments wherein the fusion sequences are transcribed but not translated, the expression constructs contain recombinant or genetically engineered target-reporter fusion sequences operably linked to elements necessary for proper transcription of the fusion sequences within the chosen host cells, including a promoter and a polyadenylation signal. In embodiments wherein the fusion sequences are transcribed and translated, the expression constructs contain recombinant or genetically engineered target-reporter fusion sequences operably linked to elements necessary for proper transcription and translation of the fusion sequences within the chosen host cells, including a promoter, a translation initiation signal (“start” codon), a translation termination signal (“stop” codon) and a polyadenylation signal. In embodiments wherein the fusion sequences encode a singe bicistronic transcript for independent translation of the target gene sequences and reporter sequences, the expression constructs additionally contain an internal ribosomal entry site (IRES) element between the target gene sequences and the reporter sequences of the target-reporter fusion.

The promoter sequences may be endogenous or heterologous to the host cell, and may provide ubiquitous (i.e., expression occurs in the absence of an apparent external stimulus and is not cell-type specific) or tissue-specific (also known as cell-type specific) expression.

Promoter sequences for ubiquitous expression may include synthetic and natural viral sequences (e.g., human cytomegalovirus immediate early promoter (CMV; Karasuyama, et al. J. Exp. Med. 1989;169:13); simian virus 40 early promoter (SV40; Bernoist, et al. Nature 1981;290:304-310; Templeton, et al. Mol. Cell Biol. 1984;4:817; and Sprague, et al. J. Virol. 1983;45:773); Rous sarcoma virus (RSV; Yamamoto, et al. Cell 1980;22:787-797); or adenovirus major late promoter), which confer a strong level of transcription of the nucleic acid molecule to which they are operably linked. The promoter can also be modified by the deletion and/or addition of sequences, such as enhancers (e.g., a CMV, SV40, or RSV enhancer), or tandem repeats of such sequences. The addition of strong enhancer elements may increase transcription by 10-100 fold.

Promoters/enhancers which may be used to control expression also include, but are not limited to, the human beta-actin promoter (Gunning, et al. Proc. Natl. Acad. Sci USA 1987;84:4831-4835), the glucocorticoid-inducible promoter present in the mouse mammary tumor virus long terminal repeat (MMTV LTR; Klessig, et al. Mol. Cell Biol. 1984;4:1354-1362), the long terminal repeat sequences of Moloney murine leukemia virus (MuLV LTR; Weiss, et al. RNA Tumor Viruses. (Cold Spring Harbor Laboratory, Cold Spring Harbor, N.Y.:1985), the herpes simplex virus (HSV) thymidine kinase promoter/enhancer (Wagner et al. Proc. Natl. Acad. Sci. USA 1981;82:3567-71), and the herpes simplex virus LAT promoter (Wolfe, et al. Nature Genetics 1992;1:379-384).

The expression constructs may further comprise vector sequences that facilitate the cloning and propagation of the expression constructs. A large number of vectors, including plasmid and fungal vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest. Prolonged expression of the encoded target-reporter fusion in in vitro cell culture may be achieved by the use of vectors sequences that allow for autonomous replication of an extrachromosomal construct in mammalian host cells (e.g., EBNA-I and oriP from the Epstein-Barr virus).

For example, a plasmid is a common type of vector. A plasmid is generally a self-contained molecule of double-stranded DNA, usually of bacterial origin, that can readily accept additional foreign DNA and which can readily be introduced into a suitable host cell. A plasmid vector generally has one or more unique restriction sites suitable for inserting foreign DNA. Examples of plasmids that may be used for expression in prokaryotic cells include, but are not limited to, pBR322-derived plasmids, pEMBL-derived plasmids, pEX-derived plasmids, pBTac-derived plasmids, and pUC-derived plasmids.

A number of vectors exist for expression in yeast. For instance, YEP24, YIP5, YEP51, YEP52, pYES2, and YRP17 are cloning and expression vehicles useful in the introduction of genetic constructs into S. cerevisiae (see, e.g., Broach, et al. “Experimental Manipulation of Gene Expression.” ed. M. Inouye (Academic Press: 1983)). These vectors can replicate in E. coli due the presence of the pBR322 ori, and in S. cerevisiae due to the replication determinant of the yeast 2 micron plasmid.

A number of expression vectors exist for expression in mammalian cells. Many of these vectors contain prokaryotic sequences to facilitate the propagation of the vector in bacteria, and one or more eukaryotic transcription regulatory sequences that cause expression in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo, and pHyg derived vectors are examples of mammalian expression vectors suitable for transfection of eukaryotic cells. Some of these vectors are modified by the addition of sequences from bacterial plasmids, such as pBR322, to facilitate replication and drug resistance selection in both prokaryotic and eukaryotic cells. Derivatives of viruses such as the bovine papilloma virus (BPV-1), or Epstein-Barr virus (pHEBo, pREP-derived and p205) may be used for transient expression of proteins in eukaryotic cells. A baculovirus expression system (see, e.g., “Current Protocols in Molecular Biology.” eds. Ausubel et al. (John Wiley & Sons:1992)) may also be used. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

For other suitable expression systems for both prokaryotic and eukaryotic cells, as well as general recombinant procedures, see “Molecular Cloning A Laboratory Manual. 2nd Edition.” Sambrook, et al. (Cold Spring Harbor Laboratory Press: 1989) Chapters 16 and 17.

The major time constraint in the screening method of the invention is imposed by the necessity of cloning each unique target-reporter fusion expression construct. This time constraint may be overcome by the use of a technique in which open reading frames are generated in universal entry clones and then transfered to destination expression vectors containing fluorescent or enzymatic reporters (Simpson, et al. EMBO Rep. 2000;1, 287-92). This technique is based upon a novel technology that circumvents traditional restriction digestion and ligation steps of cloning, namely the Gateway™ cloning system (Life Technologies). This technique provides for high-capacity cloning and expression of target gene sequences that is rapid, efficient, directional, and compatible with a range of expression vectors. This technique is summarized below.

First, primers for the target gene sequences to be cloned are designed so as to minimize primer dimer formation and hybridization to secondary sites. The target gene sequences are then amplified by PCR and recombined into an entry vector, as per manufacturer's instructions (Gateway™ cloning system: Life Technologies). The sequence inserted into the entry vector is verified by sequencing. Then, identical copies of the target gene sequence can be further cloned (again by recombination) into a wide variety of compatible Gateway™ expression vectors that already contain fluorescent protein reporter sequences. Thereafter, the target-reporter fusion sequence may be expressed by the vector as a fluorescent fusion protein (e.g., a CFP N-terminal fusion or YFP C-terminal fusion).

### Assay Systems

The RNAi probes and target-reporter fusion expression constructs of the invention are transfected into target cells, such that the target-reporter fusion is ectopically expressed when RNAi-mediated suppression of such expression is not activated. In exemplified embodiments the RNAi probes and target-reporter fusion expression constructs are introduced into in vitro cultured mammalian cell lines. Protocols for in vitro culture of mammalian cells are well established in the art: see for example, Masters, J., ed. Animal Cell Culture: A Practical Approach 3^{rd} Edition. (Oxford University Press) and Davis, J. M., ed. Basic Cell Culture 2^{nd} Edition (Oxford University Press:2002). Exemplary in vitro cultured mammalian cell lines in accordance with the present invention include human HeLa cells and murine C2C12 cells.

Techniques for introduction of nucleic acids to cells are well established in the art, including, but not limited to, electroporation, microinjection, liposome-mediated transfection, calcium phosphate-mediated transfection, or virus-mediated transfection (see, for example, Artificial self-assembling systems for gene delivery. Felgner, et al., eds. (Oxford University Press: 1996); Lebkowski, et al. Mol Cell Biol 1988;8:3988-3996; “Molecular Cloning: A Laboratory Manual.” 2^{nd} Sambrook, et al. (Cold Spring Harbor Laboratory: 1989); and “Current Protocols in Molecular Biology” Ausubel, et al., eds. (John Wiley & Sons:1989)). Various reagents and kits for introduction of nucleic acid sequences into cells are commercially available: for example, the Effectene transfection kit from Qiagen, Lipofectamine 2000 reagents from Invitrogen, and Lipofectamine PLUS reagents from Life Technologies.

In a specific embodiment, the RNAi probe and target-reporter fusion expression construct are introduced into the target cell simultaneously. However, the invention also contemplates embodiments wherein the RNAi probe and target-reporter fusion expression construct are sequentially introduced into the target cells. In one embodiment the RNAi probe is introduced into the target cell, and thereafter the target-reporter fusion expression construct is introduced into the target cell. In an alternative embodiment, the target-reporter fusion expression construct is introduced into the target cell, and thereafter the RNAi probe is introduced into the target cell. This latter embodiment contemplates development of a specialized cell line modified to stably express a target-reporter fusion. In such cells, the target-reporter fusion expression construct may be chromosomally integrated. Thus it may be possible to generate and use a cell line with stable target-reporter fusion expression for multiple assays at different time points.

Where the target-reporter fusion expression construct is introduced to a target cell prior to introduction of the RNAi probe, and the target cell is an in vitro cultured cell line, the target-reporter fusion expression construct may be used to generate a transiently or stably transfected cell line. Where the target-reporter fusion expression construct is used to generate a transiently transfected cell line, the RNAi probe must be introduced to perform the screening assay during the time frame in which the target-reporter fusion expression construct is maintained and expressed within the target cell. Where the target-reporter fusion expression construct is used to generate a stably transfected cell line, the cells may be cultured and/or stored (e.g., by freezing) for extended time periods prior to introduction of RNAi probes to perform the screening assay.

Where stable transfection of the target cell lines is desired, the introduced target-reporter fusion expression construct DNA preferably comprises linear DNA, free of vector sequences, as prepared from the target-reporter fusion expression constructs of the invention. Stably transfected in vitro cell lines may be screened for integration and copy number of the target-reporter fusion expression construct. For such screening, the genomic DNA of a cell line is prepared and analyzed for incorporation of the expression construct DNA by PCR and/or Southern blot.

### High-Throughput Screening Methods

The screening method of the present invention may be performed as a high-throughput screen. Such high-throughput methods are suitable for concurrent screening of a large number of different RNAi candidates to identify RNAi probes of desired efficacy (e.g., RNAi probes that completely abolish target gene expression or RNAi probes that reduce target gene expression by about 50%). Such high-throughout methods are also suitable for dose-response tests (concurrent screening of a large number of varying concentrations) of a given RNAi probe to identify the RNAi probe concentration that provides the desired efficacy (e.g., RNAi probe concentration that completely abolishes target gene expression or RNAi probe concentration that reduces target gene expression by about 50%). In this respect, high-throughput methods are advantagous in that the described screening (of individual RNAi candidates) and dose-response analyses (of varying concentrations of a given RNAi probe) may be performed in a single high-throughput assay.

For such high-throughput assays, RNAi probes and target-reporter gene fusion expression constructs are introduced into cells in a microarray format, and then the microarray is scored for reporter gene expression. For example, solutions containing RNAi probes and target-reporter fusion expression constructs may be placed into individual wells of a microtitre dish as an ordered array and transfected into target cells plated into the microtitre dish.

Expression of the reporter in the cells of a microarray (e.g., in wells of a microtitre dish) can be scored by standard high-throughput detection techniques (e.g., ELISA; autoradiography; or fluorescence, spectrophotometric, or chemiluminescent scanning, etc.). Commercially available scanners suitable for high-throughput visualization and quantitaion of fluorescence microtitre dish assays include, but are not limited to, ScanArray 5000 (GSI Lumonics) and the ViewLux™ ultraHTS Microplate Imager (1536-well microtitre dish format, PerkinElmer). Commercially available scanners suitable for high-throughput scanning and quantitation of chemiluminescent or spectrophotometric microtitre dish assays include, but are not limited to, the Fusion™ Universal Microplate Analyzer (6 to 1536 well microtitre dish formats, PerkinElmer) and the EnVision™ multilabel plate reader (1 to 1536 well microtitre dish formats, PerkinElmer).

The results of such detection are then analyzed to determine which RNAi probes and/or probe concentrations provide the desired degree of suppression of target gene expression. For example, the Image Quant (Fuji) software package may be used to quantitate and analyze fluorescent reporter signal intensity of transfected cells in each well of a microtitre dish. Many of the commercially available scanners integrate quantitation and data analysis into a single function performed by the scanner (e.g., the ImageTrak™ Epi-Fluorescence System from PerkinElmer).

A preferred method for high-throughput screening of RNAi probes (see, e.g., Example 5) uses a high density “reverse transfection” method described in Ziauddin and Sabatini Nature 2001;411:107-110. In this method, nucleic acids to be introduced into a cell are printed on a slide in a carrier solution (e.g., gelatin or lipid) to form a microarray. Where gelatin is used as the carrier, the gelatin solution is preferably prepared by dissolving the gelatin in water at 60° C. for 15 minutes in order to minimize variability in the quality of the gelatin solution (e.g., as caused by varying extents of gelatin degradation). The plated microarray is then preincubated with a transfection agent (e.g., Lipofectamine), and then overlaid with cells in tissue culture suspension. The cells are then allowed to grow on the microarray. Cells growing in close proximity to the printed nucleic acids will become transfected. Using fully automated liquid-dispensing and plate handling robotic systems and modern microarrays, it is possible to print nucleic acid mixtures at densities of up to 6,000 to 10,000 features per slide. Expression of reporter within transfected cells in the printed microarray is then quantitated and analyzed.

### EXAMPLES

The present invention is next described by means of the following examples. However, the use of these and other examples anywhere in the specification is illustrative only, and in no way limits the scope and meaning of the invention or of any exemplified form. Likewise, the invention is not limited to any particular preferred embodiments described herein. Indeed, many modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

### Example 1

### Synthesis of RNAI Candidates and Probes

Various siRNA and shRNA candidates, as well as siRNA and shRNA non-specific control probes were screened in the present invention. For the specific siRNA and shRNAs, the candidates were designated with respect to the translation initiation codon of the specific target gene, where the “A” of the start “ATG” is designated as position 1, and where the designation number indicates the most 5′ nucleotide of the target gene sequence that is specifically targeted by the siRNA. Designations are relative to mouse myoD (Genbank Accession # M84918) and human lamin A/C (Genbank Accession # NM_{—}005572) cDNA sequences.

Chemical synthesis of siRNAs. A custom synthetic siRNA designated Lamin A/C 608 (see Table 1) was purchased from Dharmacon Research (Lafayette, Colo.). This siRNA was provided by Dharmacon as precipitated purified duplex with a purity greater than 97%. The siRNA pellet was re-dissolved in water for use in transfection

In vitro transcription of siRNAs. Alternatively, siRNAs were synthesized by in vitro transcription essentially as described in Donze, et al. Nucleic Acids Res. 2000;30:e46. The siRNAs produced by this method are shown in Table 1. The desalted DNA oligonucleotides used for in vitro transcription of siRNA probes are shown in Table 2. Throughout Table 2, the T7 primer sequence is in italics, and the target gene-specific sequence is underlined.

For example, EGFP specific (EGFP-SP) and non-specific (NON-SP) siRNAs were synthesized. The EGFP specific (EGFP-SP) siRNA sequence is known to efficiently suppress EGFP reporter gene expression via the RNAi pathway (see Caplen, et al. Proc. Natl. Acad. Sci. USA 2001;98:9742-9747). The non-specific siRNA (NON-SP) is a scrambled sequence used as a negative control. For synthesis, the following desalted DNA oligonucleotides were ordered from Sigma Genosys (Texas):
(i) T7: 5′ TAA TAC GAC TCA CTA TAG 3′ (SEQ ID NO: 2);
(ii) EGFP sense: 5′ ATG AAC TTC AGG GTC AGC TTG CTA TAG TGA GTC GTA TTA 3′ (SEQ ID NO: 3) where the EGFP-specific sequence is underlined, and the T7 promoter sequence is in italics;
(iii) EGFP antisense: 5° CGG CAA GCT GAC CCT GAA GTT CTA TAG TGA GTC GTA TTA 3′ (SEQ ID NO: 4) where the EGFP-specific sequence is underlined, and the T7 promoter sequence is in italics;
(iii) Non-specific sense: 5′ ATG ATA CTC GAG GGC ATG TCT CTA TAG TGA GTC GTA TTA 3′ (SEQ ID NO: 5) where the scrambled non-specific sequence is underlined, and the T7 promoter sequence is in italics; and
(iv) Non-specific antisense: 5° CGG AGA CAT GCC CTC GAG TAT CTA TAG TGA GTC GTA TTA 3′ (SEQ ID NO: 6) where the scrambled non-specific sequence is underlined, and the T7 promoter sequence is in italics.

The oligonucleotide-directed production of small RNA transcripts with T7 RNA polymerase was performed essentially as described (Milligan and Uhlenbeck. Methods Enzymol. 1989;180:51-62). For each transcription reaction, 1 nmol of T7 oligonucleotide was mixed with 1 nmol of a sense or antisense oligonucleotides in 501A1 of TE buffer (10 mM Tris-HCl pH8.0, and 1 mM EDTA) and then heated at 95° C. After 2 min at 95° C., the heating block was switched off and allowed to slowly cool to room temperature to obtain the annealed template. Transcription was performed in 50 μl of transcription mix (40 mM Tris-HCl pH7.9, 6 mM MgCl₂, 10 mM DTT, 10 mM NaCl, 2mM spermidine, 1 mM rNTPs, 0.1 Units yeast pyrophosphatase (Sigma), 40 Units RNaseOUT (Life Technologies) and 100 Units T7 RNA polymerase (Fermentas) containing 200 pmol of the annealed template. After incubation at 37° C. for 2 hr, 1 Unit RNase free-DNase (Promega) was added and the reaction was incubated at 37° C. for 15 min.

Thereafter, sense and antisense 22 nt RNAs generated in separate transcription reactions were annealed by mixing both crude transcription reactions, and incubating the mixture first at 95° C. for 5 min and then at 37° C. for 1 hr. This mixture of annealed T7 RNA polymerase synthesized small interfering double-stranded RNA (100 μl) was then adjusted to 0.2M sodium acetate pH5.2, and precipitated with 2.5 volumes ethanol. After centrifugation, the pellet was washed once with 70% ethanol, dried, and resuspended in 50 μl of water for use in transfections.

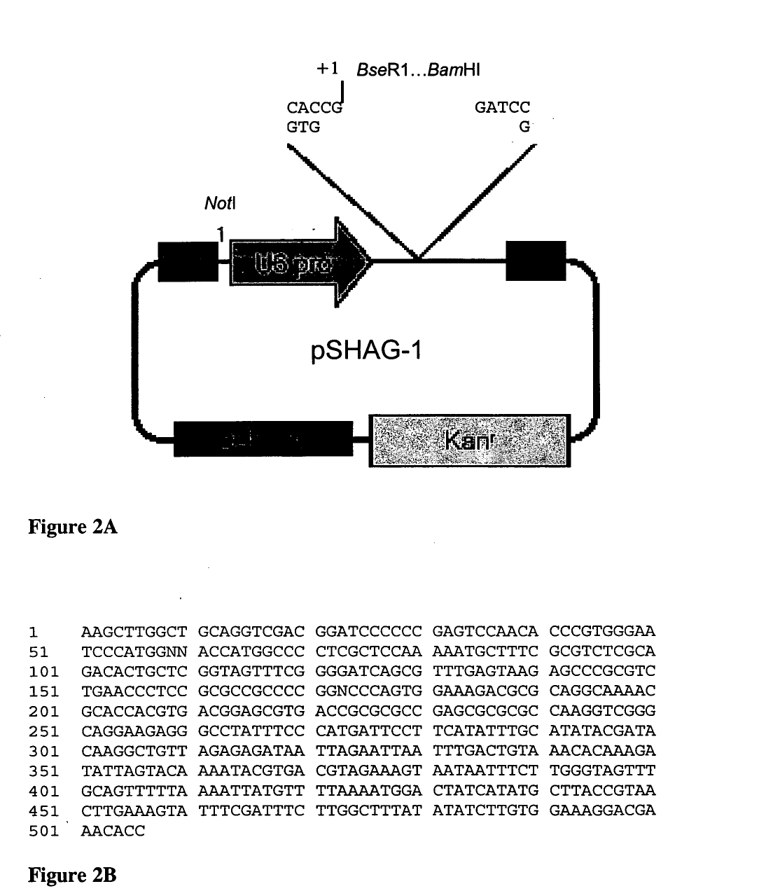

Constructs for in vivo expression of shRNAs. For the MyoD-sepcific shRNA expression constructs, double stranded DNA fragments encoding shRNA sequences were cloned directly into a U6 promoter-containing vector, pSHAG-1 (FIG. 2A). pSHAG-1 is a derivative of the pENTR/D-TOPO vector (Invitrogen) in which a 506 bp segment of the human U6 promoter (FIG. 2B; SEQ ID NO: 1) and linker sequences containing BseRI and BamHI restriction sites have been inserted into the NotI site of pENTR/D-TOPO.

To assemble the MyoD-specific shRNA expression constructs, two complementary DNA oligomers of about 73 nucleotides were ordered from Sigma Genosys. These oligomers were then annealed to form a double stranded DNA (dsDNA) fragment with overhanging single stranded regions complementary to the BseRI and BamHI overhangs of linear BseRI and BamHI digested pSHAG-1 vector (see Table 3 and FIG. 2A). The target-gene specific sequence of these inserts is indicated by underlining in Table 3.

These annealed dsDNAs were then ligated to linear BseRI and BamHI digested pSHAG-1 vector to create the shRNA expression constructs. The 3′-most “G” residue of the BseRI site overhang represents the +1 site for transcription initiation in these constructs.

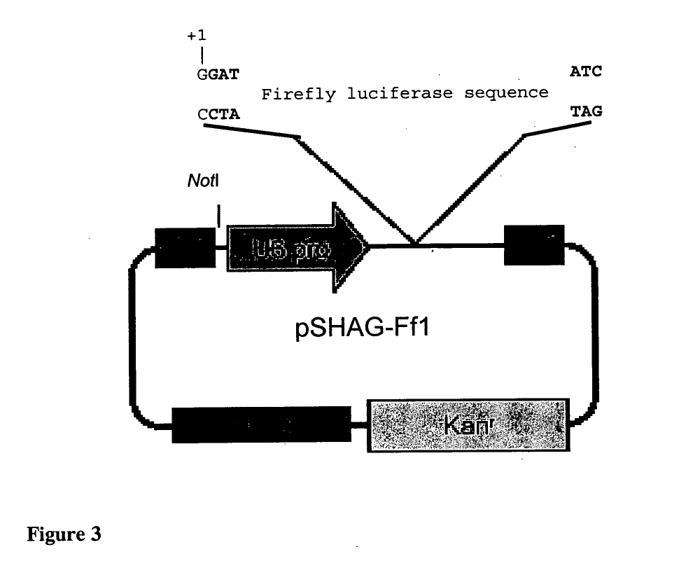

An shRNA expression plasmid encoding a Firefly luciferase-specific shRNA was used as a non-specific shRNA control (NON-SP shRNA, see Table 3). For this NON-SP shRNA expression construct, double stranded DNA fragments encoding Firefly luciferase-specific shRNA sequences were cloned directly into a U6 promoter-containing vector, pSHAG, to create pSHAG-Ff1 (FIG. 3 and Table 3). pSHAG is a derivative of the pENTR/D-TOPO vector (Invitrogen) in which a 506 bp segment of the human U6 promoter (FIG. 2B; SEQ ID NO: 1) and linker sequences containing an EcoRV restriction site have been inserted into the NotI site of pENTR/D-TOPO. To assemble the non-specific shRNA expression construct, two complementary DNA oligomers of about 73 nucleotides were ordered from Sigma Genosys. These oligomers were then annealed to form a blunt-ended double stranded DNA (dsDNA) fragment (See Table 3). This annealed dsDNAs was then ligated to linear EcoRV digested pSHAG vector to create pSHAG-Ff1. The vector sequence G residue immediately 5′ of the EcoRV half-site into which the dsDNA fragment is inserted represents the +1 site for transcription initiation in this construct.

The assembled shRNA expression constructs were then transformed into target cells to provide in vivo expression of the shRNAs.

TABLE 1
siRNA probes

| | | | |
|---|---|---|---|
| siRNA Probe | Designation | Sequence | SEQ ID NOs |

| | | | |
|---|---|---|---|
| EGFP-specific siRNA | EGFP-SP | 5′-GCAAGCUGACCCUGAAGUUCAU-3′ | SEQ ID NO:7 |
| | | \|\|\|\|\|\|\|\|\|\|\|\|\|\|\|\|\|\|\|\| | and |
| | | 3′-GCCGUUCGACUGGGACUUCAAG-5′ | SEQ ID NO:8 |
| Non-specific siRNA | NON-SP | 5′-GAGACAUGCCCUCGAGUAUCAU-3′ | SEQ ID NO:9 |
| (control) | | \|\|\|\|\|\|\|\|\|\|\|\|\|\|\|\|\|\|\|\| | and |
| | | 3′-GCCUCUGUACGGGAGCUCAUAG-5′ | SEQ ID NO:10 |
| MyoD-specific | 25 | 5′-GGCCUGUCAAGUCUAUGUCCC-3′ | SEQ ID NO:11 |
| | | \|\|\|\|\|\|\|\|\|\|\|\|\|\|\|\|\| \| | and |
| | | 3′-CCCCGGACAGUUCAGAUACGG | SEQ ID NO:12 |
| | 294 | 5′-GGUCUUGCGCUUGCACGCCUU-3′ | SEQ ID NO:13 |
| | | \|\|\|\|\|\|\|\|\|\|\|\|\|\|\|\|\|\|\| | and |
| | | 3′-CACCAGAACGCGAACGUGCGG-5′ | SEQ ID NO:14 |
| | 438 | 5′-GGCGUUGCGCAGGAUCUCCAC-3′ | SEQ ID NO:15 |
| | | \|\|\|\|\|\|\|\|\|\|\|\|\|\|\|\|\|\|\| | and |
| | | 3′-UACCGCAACGCGUCCUAGAGG-5′ | SEQ ID NO:16 |
| | 538 | 5′-GGCCUGGGGGCAGCGGUCCAG-3′ | SEQ ID NO:17 |
| | | \|\|\|\|\|\|\|\|\|\|\|\|\|\|\|\|\|\|\| | and |
| | | 3′-UGCCGCGGACCCCCGUCGCCAGG-5′ | SEQ ID NO:18 |
| | 637 | 5′-GGGGGCCGCUUGGGGGGCCGC-3′ | SEQ ID NO:19 |
| | | \|\|\|\|\|\|\|\|\|\|\|\|\|\|\|\|\|\|\| | and |
| | | 3′-GGCCCCCGGCGAACCCCCCGG-5′ | SEQ ID NO:20 |
| Lamin A/C-specific | −164 | 5′-GGCCGGGCGCUGUCGGACCUC-3′ | SEQ ID NO:21 |
| | | \|\|\|\|\|\|\|\|\|\|\|\|\|\|\|\|\|\|\| | and |
| | | 3′-ACCCGGCCCGCGACAGCCUGG-5′ | SEQ ID NO:22 |
| | 608 | 5′-CUGGACUUCCAGAAGAACAUCdTdT-3′ | SEQ ID NO:23 |
| | | \|\|\|\|\|\|\|\|\|\|\|\|\|\|\|\|\|\|\|\|\| | and |
| | | 3′-dTdTGACCUGAAGGUCUUCUUGUAG-5′ | SEQ ID NO:24 |
| | 787 | 5′-GGCAGAAUAAGUCUUCUCCAG-3′ | SEQ ID NO:25 |
| | | \|\|\|\|\|\|\|\|\|\|\|\|\|\|\|\|\|\|\| | and |
| | | 3′-AACCGUCUUAUUCAGAAGAGG-5′ | SEQ ID NO:26 |
| | 979 | 5′-GGUGUCCCGCUCACGGGCCAG-3′ | SEQ ID NO:27 |
| | | \|\|\|\|\|\|\|\|\|\|\|\|\|\|\|\|\|\|\| | and |
| | | 3′-GACCACAGGGCGAGUGCCCGG-5′ | SEQ ID NO:28 |
| | 1755 | 5′-GGCUGGGGAGAGGCUGCCCCC-3′ | SEQ ID NO:29 |
| | | \|\|\|\|\|\|\|\|\|\|\|\|\|\|\|\|\|\|\| | and |
| | | 3′-CUCCGACCCCUCUCCGACGGG-5′ | SEQ ID NO:30 |

TABLE 2
Primers used for in vitro transcription of siRNA probes

| | | |
|---|---|---|
| | siRNA probe | |
| Target gene | designation | Desalted DNA oligonucleotides |

| | | | |
|---|---|---|---|
| EGFP | EGFP-SP | 5′-ATGAACTTCAGGGTCAGCTTGC TATAGTGAGTCGTATTA-3′ | SEQ ID NO:3 |
| | | and | and |
| | | 5′-CGGCAAGCTGACCCTGAAGTTC TATAGTGAGTCGTATTA-3′ | SEQ ID NO:4 |
| Non specific | NON-SP | 5′-ATGATACTCGAGGGCATGTCTC TATAGTGACTCGTATTA-3′ | SEQ ID NO:5 |
| (control) | | and | and |
| | | 5′-CGGAGACATGCCCTCGAGTATC TATAGTGAGTCGTATTA-3′ | SEQ ID NO:6 |
| MyoD | 25 | 5′-GGGACATAGACTTGACAGGCC TATAGTGAGTCGTATTAC-3′ | SEQ ID NO:31 |
| | | and | and |
| | | 5′-GGGGCCTGTCAAGTCTATGCC TATAGTGAGTCGTATTAC-3′ | SEQ ID NO:32 |
| | 294 | 5′-AAGGCGTGCAAGCGCAAGACC TATAGTGAGTCGTATTAC-3′ | SEQ ID NO:33 |
| | | and | and |
| | | 5′-GTGGTCTTGCGCTTGCACGCC TATAGTGAGTCGTATTAC-3′ | SEQ ID NO:34 |
| | 438 | 5′-GTGGAGATCCTGCGCAACGCC TATAGTGAGTCGTATTAC-3′ | SEQ ID NO:35 |
| | | and | and |
| | | 5′-ATGGCGTTGCGCAGGATCTCC TATAGTGAGTCGTATTAC-3′ | SEQ ID NO:36 |
| | 538 | 5′-CTGGACCGCTGCCCCCAGGCC TATAGTGAGTCGTATTAC-3′ | SEQ ID NO:37 |
| | | and | and |
| | | 5′-ACGGCCTGGGGGCAGCGGTCC TATAGTGAGTCGTATTAC-3′ | SEQ ID NO:38 |
| | 637 | 5′-GCGGCCCCCCAAGCGGCCCCC TATAGTGAGTCGTATTAC-3′ | SEQ ID NO:39 |
| | | and | and |
| | | 5′-CCGGGGGCCGCTTGGGGGGCC TATAGTGAGTCGTATTAC-3′ | SEQ ID NO:40 |
| Lamin A/C | −164 | 5′-GAGGTCCGACAGCGCCCGGC CTATAGTGAGTCGTATTAC-3′ | SEQ ID NO:41 |
| | | and | and |
| | | 5′-TGGGCCGGGCGCTGTCGGAC CTATAGTGAGTCGTATTAC-3′ | SEQ ID NO:42 |
| | 787 | 5′-CTGGAGAAGACTTATTCTGC CTATAGTGAGTCGTATTAC-3′ | SEQ ID NO:43 |
| | | and | and |
| | | 5′-TTGGCAGAATAAGTCTTCTC CTATAGTGAGTCGTATTAC-3′ | SEQ ID NO:44 |
| | 979 | 5′-CTGGCCCGTGAGCGGGACAC CTATAGTGAGTCGTATTAC-3′ | SEQ ID NO:45 |
| | | and | and |
| | | 5′-CTGGTGTCCCGCTCACGGGC CTATAGTGAGTCGTATTAC-3′ | SEQ ID NO:46 |
| | 1755 | 5′-GGGGGCAGCCTCTCCCCAGC CTATAGTGAGTCGTATTAC-3′ | SEQ ID NO:47 |
| | | and | and |
| | | 5′-GAGGCTGGGGAGAGGCTGCC CTATAGTGAGTCGTATTAC-3′ | SEQ ID NO:48 |

TABLE 3
shRNA expression construct dsDNA inserts

| | |
|---|---|
| Desig- | |
| nation | Inserted double stranded DNA sequence |

Non-specific shRNA (control)

| | | |
|---|---|---|
| NON-SP | 5′-TCCAATTCAGCGGGAGCCACCTGATGAAGCTTGATCGGGTGGCTCTCGCTGAGTTGGAATCCATTTTTTTT-3′ | SEQ ID NO:49 |
| shRNA | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | and |
| | 3′-AGGTTAAGTCGCCCTCGGTGGACTACTTCGAACTAGCCCACCGAGAGCGACTCAACCTTAGGTAAAAAAAA-5′ | SEQ ID NO:50 |

MyoD-specific

| | | |
|---|---|---|
| 1 | 5′-TCCCGGAGTGGCGGCGATAGAAGCTCCAGAAGCTTGTGGAGCTTCTGTCGCCGCCGCTTCGGGATATTTTTTT-3′ | SEQ ID NO:51 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | and |
| | 3′-GCAGGGCCTCACCGCCGCTATCTTCGAGGT CTTCGAACACCTCGAAGACAGCGGCGGCGAAGCCCTATAAAAAAAC | SEQ ID NO:52 |
| | TAG-5′ | |
| 312 | 5′-CGGCCTTGCGGCGATCAGCGTTGGTGGTGAAGCTTGATCACCAGCGCTGGTCGCCGCAAGGTCGCCATTTTTT-3′ | SEQ ID NO:53 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | and |
| | 3′-GCGCCGGAACGCCGCTAGTCGCAACCACCA CTTCGAACTAGTGGTCGCGACCAGCGGCGTTCCAGCGGTAAAAAAC | SEQ ID NO:54 |
| | TAG-5′ | |
| 507 | 5′-CGTAGAAGGCAGCGGCGCCAGGGGGCGCGAAGCTTGGTGCCCCTTGGCGTCGCTGTCTTCTACGCACTTTTTT-3′ | SEQ ID NO:55 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | and |
| | 3′-GCGCATCTTCCGTCGCCGCGGTCCCCCGCG CTTCGAACCACGGGGAACCGCAGCGACAGAAGATGCGTGAAAAAAC | SEQ ID NO:56 |
| | TAG-5′ | |
| 708 | 5′-ACACAGCCGCACTCTTCCCTGGCCTGGAGAAGCTTGTTCAGGCTAGGGAGGAGTGTGGCTGTGTCGATTTTTT-3′ | SEQ ID NO:57 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | and |
| | 3′-GCTGTGTCGGCGTGAGAAGGGACCGGACCT CTTCGAACAAGTCCGATCCCTCCTCACACCGACACAGCTAAAAAAC | SEQ ID NO:58 |
| | TAG-5′ | |
| 897 | 5′-AGCCTGCAGGACACTGAGGGGCGGCGTCGAAGCTTGGGCGCCGTCCCTCGGTGTCTTGCAGGCTCAATTTTTT-3′ | SEQ ID NO:59 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | and |
| | 3′-GCTCGGACGTCCTGTGACTCCCCGCCGCAG CTTCGAACCCGCGGCAGGGAGCCACAGAACGTCCGAGTTAAAAAAC | SEQ ID NO:60 |
| | TAG-5′ | |

### Example 2

### Target-Reporter Fusion Protein and Control Expression Constructs

This Example describes the assembly of various target-reporter fusion expression contructs. In all of the following examples the target-reporter fusion sequences encode a target-reporter fusion protein produced by translation of target gene and reporter sequences that were fused so as to maintain the translational frame established by a single 5′ translation intitiation sequence. For all constructs, the integrity of sequences encoding the target-reporter fusion, and the orientation of the target gene with respect to the reporter gene within these sequences, was confirmed by restriction enzyme digestion and DNA sequencing.

pDsRed2-N1, pEGFP-N2, and pRluc-N3. Plasmids pDsRed2-N1 and pEGFP-N2 (Genbank Accession # U57608) are both available from Clontech (Clontech Inc., Palo Alto, Calif.). Plasmid pRluc-N3 is available from Perkin Elmer (PerkinElmer, Boston, Mass.).

EGFP-RFP fusion construct. The red fluorescent protein (RFP) cDNA was amplified from pDsRed2-N1 by PCR under standard conditions and cycling parameters using the primers RFP-1 (5′-TTT TTG GAT CCC ATA CAG GAA CAG GTG GTG-3′; SEQ ID NO: 61) and RFP-2 (5′-CGC CAG CAA CAA CGC GGC CTT TTT AC-3′; SEQ ID NO: 62). This RFP PCR product was digested with BamH 1, and ligated with BamHI digested pEGFP-N2 to form EGFP-RFP, in which the RFP sequences are linked to the 3′ end of the EGFP sequences.

RFP-EGFP fusion construct. The EGFP cDNA was amplified from pEGFP-N2 by PCR under standard conditions and cycling parameters using the primers EGFP-1 (5′-TTT TGG ATC CCG ATA CTT GTA CAG CTC GTC-3′; SEQ ID NO: 63) and EGFP-2 (5′-CGC CAG CAA CAA CGC GGC CTT TTT AC-3′; SEQ ID NO 64). This EGFP PCR product was digested with BamHI, and ligated with BamHI digested pDsRed2-N1 to form RFP-EGFP, in which the EGFP sequences are linked to the 3′ end of the RFP sequences.

EGFP-Rluc fusion construct. The EGFP cDNA was amplified from pEGFP-N2 by PCR under standard conditions and cycling parameters using the primers EGFP-1 (5′-TTT TGG ATC CCG ATA CTT GTA CAG CTC GTC-3′; SEQ ID NO: 63) and EGFP-2 (5′-CGC CAG CAA CAA CGC GGC CTT TTT AC-3′; SEQ ID NO 64). This EGFP PCR product was digested with BamH1, and ligated with BamHI digested pRluc-N3 to form RFP-EGFP, in which the Renilla Luciferase sequences are linked to the 3′ end of the RFP sequences.

MyoDEGFP fusion construct. The Mus musculus MyoD cDNA (Genbank Accession # M84918) was amplified from the plasmid pCMV-MyoDs by PCR under standard conditions and cycling parameters using the primers MyoD-1 (5′-TTT TCT C GAG ATG GAG CTT CTA TCG CCG-3′; SEQ ID NO: 65) and MyoD-2 (5′-GTG GAT CCC ACA AAG CAC CTG ATA AAT-3′; SEQ ID NO: 66). Plasmid pCMV-MyoDs contains the 1785 bp EcoRI fragment of the MyoD cDNA ligated into the EcoRI site of the expression plasmid pCSA (Cytomegalovirus promoter/SV40 Splica & polyA sites with ampicillin resistance).

The MyoD PCR product was digested with XhoI and BamH1, and ligated with XhoI and BamHI digested pEGFP-N2 to form MyoD-EGFP, in which the EGFP sequences are linked to the 3′ end of the MyoD sequences.

EGFP-lamin A/C fusion construct. The pEGFP-N2 vector was digested with BsrGI and NotI and filled in by T4 DNA polymerase. The Not I and Sal I fragment of human Lamin A/C (Genbank Accession # NM_{—}005572) was obtained by digestion of a Lamin A/C-pSPORT I vector (Research Genetics). These two blunt-end fragments were ligated to form EGFP-Lamin A/C, in which the Lamin A/C sequences are linked to the 3′ end of the EGFP sequences.

### Example 3

### Validation of the Target-Reporter Fusion Construct System

The feasibility of the experimental design was tested by evaluating critical parameters associated with the target-reporter fusion products, such as stability of fusion proteins, accessibility of target site in the chimeric mRNA, and specificity of siRNA probes in suppressing cognate gene expression as reflected by changes in reporter expression. Taken together the data indicate that the target-reporter fusion products are stable, and that the target site in the fusion mRNA is accessible for specific siRNA mediated gene suppression in both 3′end or 5′end target-reporter fusions (i.e., where the fusion sequence is organized as 5′-target-reporter-3′ or 5′-reporter-target-3′). The latter property is particularly attractive, since it allows for substantial flexibility in the construction of fusion constructs. Furthermore, these experiments showed that siRNA-mediated suppression of target gene expression is faithfully reported by the reporter to which the target gene is fused, and that the effect of siRNAs probes on target gene and reporter expression is dose dependent.

Fluorescent reporter genes. To test the fluorescent reporter-based system, enhanced green fluorescent protein (EGFP) and red fluorescent protein (RFP) were used as target gene and reporter gene, respectively. An EGFP-specific siRNA (EGFP-SP) and a non-specific control siRNA (NON-SP) were generated as described in Example 1 above.

The plasmid pEGFP-N2 (Clontech Inc., Palo Alto, Calif.: Genbank Accession # U57608), in which expression of EGFP is driven by a constitutive human cytomegalovirus (CMV) immediate early promoter, was used to provide expression of EGFP transcript and protein. The plasmid pDsRed-N1 (Clontech Inc., Palo Alto, Calif.), in which the expression of RFP is driven by a constitutive human cytomegalovirus (CMV) immediate early promoter, was used to provide expression of RFP transcript and protein.

For the screening assay, 10 ng of pEGFP-N2, 50 ng of pDsRed-N1, and either EGFP-specific siRNA (2 μg) or non-specific siRNA (2 kg) were co-transfected into murine C2C12 cells (ATCC # CRL-1772). The cells were cultured in DMEM supplemented with 10% FBS, 100 U/ml penicillin and 100 μg/ml streptomycin (Life Technologies, Rockville, Md.) at 37° C. in a 5% C02-humidified chamber. Cells were transfected in 6-well plates at 60-70% confluence using Lipofectamine PLUS (Life Technology, Calif.).

Transfection was performed according to manufacturers instructions. The cells were plated on the dish in 0.5 ml of DMEM supplemented with 10% FBS (no antibiotics). For each well of cells to be transfected, the nucleic acids were diluted into 50 μl of OPTI-MEM®I Reduced Serum Medium without serum. Then for each well of cells, 1.5 μl of LIPOFECTAMINE 2000 (LF2000™) Reagent was mixed with 50 μl OPTI-MEMI Medium and incubated for 5 min at room temperature. The diluted LF2000 Reagent and diluted nucleic acids were then combined. Note that once the LF2000 Reagent is diluted, it is combined with the diluted nucleic acids within 30 min. because longer incubation times may result in decreased activity. The LF2000 and nucleic acid mixtue was then incubated at room temperature for 20 min to allow LF2000 Reagent-nucleic acid complexes to form. Then the DMEM supplemented with 10% FBS was removed from the plated cells, and replaced with 0.5 mL of fresh DMEM without FBS. The LF2000 Reagent-nucleic acid complexes (100 μl total volume) was then added to each well, and the medium mixed gently by rocking the plate back and forth. The cells were incubated at 37° C. in a CO₂ incubator for 4-5 h. Then 0.5 ml of DMEM supplemented with 20% FBS was added to each well (for a final concentration of 10% FBS), and the cells incubated at 37° C. in a CO₂ incubator.

In some instances, cells were stained 24 hours post-transfection with DAPI (4′, 6′-diamidino-2-phenylindole hydrochloride, available from Sigma), where DAPI images served as a positive control for cell number and density. DAPI staining was performed as follows: the cells were (1) washed once with PBS; (2) fixed with 70% EtOH for 20 min at room temperature; (3) washed once with PBS; (4) incubated in 1 μg/ml DAPI for 12 minutes at room temp; and (5) washed once PBS.

24 hours post-transfection, EGFP, RFP, and DAPI images were captured using a Zeiss AxioCam HRm camera at equal exposure time. Excitation wavelenghts and band pass filter wavelengths, respectively, for each image were as follows: for EGFP 490 nm and 525 nm; for RFP 596 nm and 615 nm; and for DAPI 350 nm and 470 nm.

When murine C2C12 cells were co-transfected with EGFP-specific siRNA (EGFP-SP) and two independent expression constructs for EGFP (pEGFP-N2) and RFP (pDsred2-N1), a significant reduction in EGFP expression but not in RFP expression was observed demonstrating efficacy and specificity of siRNA in suppressing expression of the target EGFP reporter gene. As expected, transfection of cells with the two plasmids and the non-specific (NON-SP) siRNA did not affect either EGFP or RFP expression.

Next, expression constructs encoding an N-terminal and a C-terminal target-reporter fusion protein (EGFP-RFP and RFP-EGFP) were both tested to determine whether siRNA against the target gene (EGFP) would result in the abrogation of reporter gene (RFP) expression. These plasmids were prepared as described in Example 2.

The EGFP-RFP or RFP-EGFP plasmid (100 ng) and either EGFP-specific siRNAs (2 μg) or non-specific siRNAs (2 μg), were co-transfected into murine C2C12 cells. The cells were cultured in DMEM supplemented with 10% FBS, 100 U/ml penicillin and 100 μg/ml streptomycin (Life Technologies, Rockville, Md.) at 37° C. in a 5% CO₂-humidified chamber. Cells were transfected in 6-well plates at 60-70% confluence using Lipofectamine PLUS (Life Technology, CA). Transfection was performed according to manufacturers instructions as described above. 24 hours post-transfection, EGFP and RFP images were captured using a Zeiss AxioCam HRm, as described above. In some cases, staining with DAPI served as a positive control for cell number and density.

When murine C2C12 cells were co-transfected with EGFP-specific siRNA (EGFP-SP) and either an EGFP-RFP fusion protein expression construct or an RFP-EGFP fusion protein expression construct, a significant reduction in both EGFP expression and RFP expression was observed. As expected, co-transfection with non-specific siRNA (NON-SP) did not affect expression of EGFP or RFP from either fusion construct. These results indicate that the siRNA-mediated suppression of target gene (EGFP) expression is faithfully reported by the reporter (RFP) to which the target gene is fused.

Enzymatic reporter genes. In addition to a fluorescent-based reporter, an enzymatic reporter was explored to demonstrate flexibility in the choice of reporter systems. Using this system the siRNA dosage effect on suppression of cognate gene expression was demonstrated. An expression construct encoding a EGFP-Renilla luciferase fusion protein (EGFP-Rluc) was prepared as described in Example 2.

The plasmid pGL3-Control (Promega: Genbank Accession # U47296), in which expression of a modified coding region for firefly (Photinus pyralis) luciferase is regulated by the SV40 (Simian Virus 40) promoter and enhancer, was used to provide expression of firefly luciferase transcript and protein. This plasmid served as an internal control for transfection efficiency.

Murine C2C12 cells were co-transfected with 300 ng EGFP-Rluc, 200 ng pGL3-Control (Promega: Genbank Accession # U47296), and increasing concentrations (12.5 ng, 25 ng, 50 ng, 100 ng, and 250 ng) of EGFP-specific siRNA (EGFP-SP) or non-specific siRNA (NON-SP). Cells were cultured in DMEM supplemented with 10% FBS, 100 U/ml penicillin and 100 μg/ml streptomycin (Life Technologies, Rockville, Md.) at 37° C. in a 5 % CO₂-humidified chamber, and transfected in 24-well plates using Lipofectamine PLUS (Life Technology, CA) according to manufacturers instructions as described above. 24 hours after transfection, EGFP images were captured using a Zeiss AxioCam HRm camera (as described above) using equal exposure time for all panels.

This analysis showed that in the range of 50-250 ng, the EGFP-SP siRNA caused a specific dose-dependent decrease in EGFP target gene expression with 250 ng required for maximal inhibition. As expected, the NON-SP siRNA had no detectable effect on EGFP expression in this range.

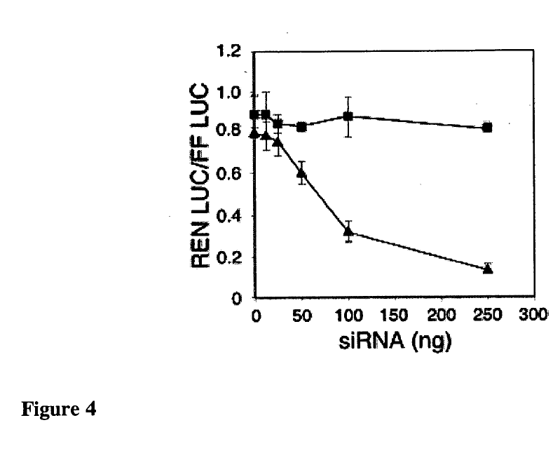

In addition, the relative amount of Renilla and firefly luciferase in transfected cells of this experiment was analyzed by a dual luciferase assay (Dual-Luciferase® Reporter Assay System: Promega) using a luminometer (Model 3010, Analytical Scientific instruments). The Renilla/Firefly luciferase ratio (REN LUC/FF LUC) was calculated and normalized against a control without siRNA (cells were transfected with neither EGFP-specific nor non-specific siRNA). These normalized REN LUC/FF LUC values were then plotted versus EGFP-SP siRNA (▴). or NON-SP siRNA (▪) concentration (see FIG. 4). This analysis showed that EGFP-SP siRNA specifically decreased Renilla luciferase reporter gene activity in a dose dependent manner consistent with the results observed for the EGFP target gene. As expected, NON-SP siRNA had no effect on Renilla luciferase reporter gene activity. An approximately 5-fold reduction in Renilla luciferase by specific siRNA relative to a control non-specific siRNA was observed.

These results indicate that siRNA-mediated suppression of the target gene (EGFP) expression is faithfully reported by the reporter (Renilla luciferase) to which the target gene is fused. Furthermore, the effect of siRNA on target gene and reporter expression is dose dependent.

### Example 4

### Identification of Effective siRNA Probes by Screening Assay

Candidate siRNAs and shRNAs for MyoD and Lamin A/C target genes were designed using computer software accessible at (http://www.cshl.org/public/SCIENCE/hannon.html).

For each of the screening assays of this example, 150 ng of siRNAs or shRNAs, 100 ng of target-reporter fusion construct, and 50 ng of pDsRed-N1 (internal control) were used to transfect murine C2C12 or human HeLa cells (ATCC # CCL-2). The cells were cultured in DMEM supplemented with 10% FBS, 100 U/ml penicillin and 100 μg/ml streptomycin (Life Technologies, Rockville, Md.) at 37° C. in a 5% CO₂-humidified chamber. Cells were transfected in 24-well plates using Lipofectamine PLUS (Life Technology, CA) per the manufacturer's instructions as described above. 24 hours post-transfection, EGFP and RFP images were captured using a Zeiss AxioCam HRm camera at equal exposure time for all panels (as described above). Cleared cell lysates were prepared from the imaged cells, and EGFP and RFP fluorescent therein quantitated using a Multilabel Counter (PerkinElmer, Boston, Mass.) with Wallac 1420 software. From these quantitated values, EGFP/RFP ratios were calculated for cells transfected with NON-SP versus EGFP-SP siRNA samples. The EGFP/RFP ratio for NON-SP siRNA cells (EGFP NON-SP/RFP NON-SP) was defined as a ratio of 1 (indicating an absence of effect). The EGFP/RFP ratios for EGFP-SP siRNA cells (EGFP EGFP-SP/RFP EGFP-SP) were then normalized based upon the normalization factor required to equate (EGFP NON-SP/RFP NON-SP) to 1. This normalization may be represented by the following formulas and computational steps: (1) (EGFP NON-SP/RFP NON-SP)×(Normalization Factor)=1; and therefore (2) (Normalization Factor)=1/(EGFP NON-SP/RFP NON-SP); then (3) solve for (Normalization Factor); and finally (4) use the calculated Normalization factor to calculate Normalized GFP/RFP for EGSP-SP siRNA cells, where Normalized GFP/RFP=(EGFP EGFP-SP/RFP EGFP-SP)×(Normalization Factor).

To demonstrate that the identified RNAi probes repressed expression of the endogenous target genes, cells were transfected with the effective RNAi probes identified by the screen. The level of endogenous target gene expression was then determined by Western Blotting performed according to standard methods (see, for example, Harlow and Lane. Antibodies: A Laboratory Manual. (Cold Spring Harbor Press, New York: 1988) using α-MyoD or α-Lamin A/C primary antibodies (Santa Cruz, Calif.). Briefly, cells were harvested at 48 hours post transfection, washed with TBS (50 mM Tris, pH8.0, 150 mM NaCl), and lysed in 100 μl of RIPA lysis buffer (TBS supplemented with 1% NP-40 and complete protease inhibitors, Roche Applied Science, Germany). Equal amounts of cell lysate were subjected to western blot analysis using α-MyoD or Lamin A/C primary antibody (Santa Cruz, Calif.). The blots were stripped with by 2 washes with 100 mM β-mercaptoethanol, 2% SDS, 62.5 mM Tris-HCl, pH 6.7 for 30 min at 50° C. for 30 min each. The stripped blots were then re-probed with anti α-tubulin (Sigma) primary antibody as a loading control (to show that approximately equal amount of protein were loaded in each lane of the gel).

MyoD. The MyoD gene was used as a prototype in this screen because of its robust expression in muscle precursor cells and the availability of reliable antibodies to monitor levels of the protein.

Five siRNAs targeting various regions spanning the MyoD coding sequence were synthesized as described in Example 1 (see Table 1). Five plasmid-encoded shRNAs targeting various regions spanning the MyoD coding sequence were synthesized as described in Example 1 (see Table 3 and FIG. 2A).

Murine C2C12 cells were co-transfected with plasmids MyoD-EGFP (prepared as described in Example 2) and dSRed2-N1 (internal control for transfection), and with individual MyoD-specific siRNA probes. 24 hours post-transfection, fluorescence microscopy images of EGFP and RFP were captured. Of the siRNAs tested, MyoD-specific siRNA 25 showed the most significant reduction in the number of EGFP positive cells when compared to cells transfected with non-specific siRNA (NON-SP).

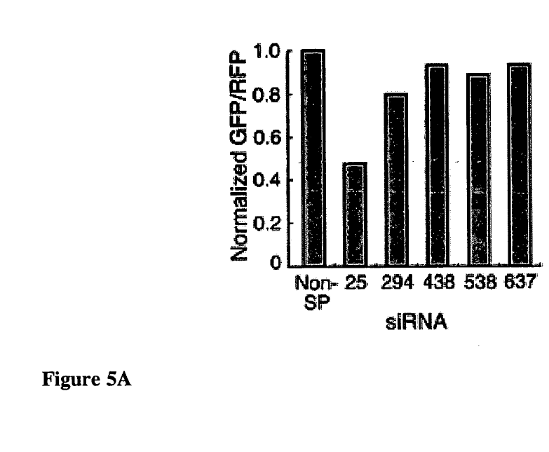

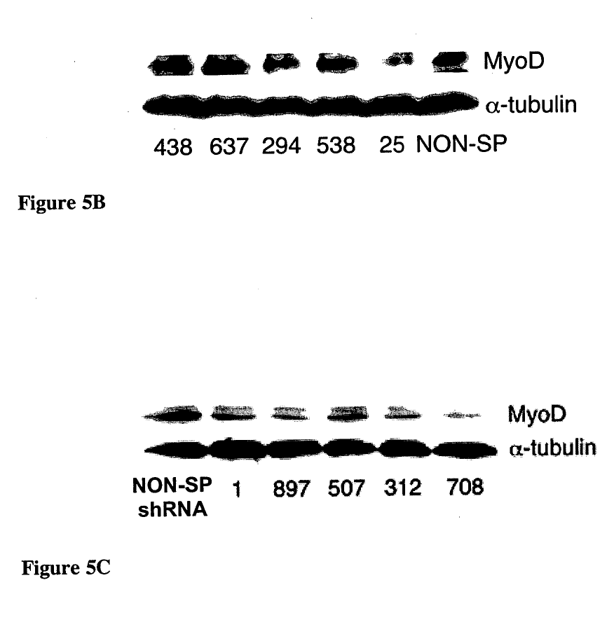

The normalized fluorescence intensity ratio (Normalized GFP/RFP) of target (MyoD-EGFP) to the internal control (RFP) was quantitated by examining protein lysates from transfected cells. The results of this analysis show that that siRNA 25 was the most effective in the suppression of ectopic MyoD-EGFP gene expression (FIG. 5A) in agreement with the microscopic imaging results.

To demonstrate the ability of the MyoD-specific siRNA to inhibit expression of both ectopically expressed MyoD-EGFP and endogenous MyoD, cells were transfected with 150 ng of siRNAs specific to MyoD and subjected to western blot analysis using an a-MyoD antibody (FIG. 5B). The results of this analysis showed a strong correlation between suppression of ectopic MyoD-EGFP and endogenous MyoD gene expression by the same panel of siRNAs, as MyoD-specific siRNA 25 showed the most significant effect to suppress endogenous MyoD.

This assay was then used to screen for effective plasmid-encoded shRNAs. Murine C2C12 cells were co-transfected with MyoD-EGFP (prepared as described in Example 2), dSRed2-N1 (internal control for transfection), and plasmid-encoded MyoD-specific shRNA probes or a non-specific shRNA probe (NON-SP shRNA). 24 hours post-transfection, fluorescence microscopy images of EGFP and RFP were captured. Of the shRNAs tested, MyoD-specific shRNA 708 showed the most significant reduction in the number of EGFP positive cells when compared to cells transfected with non-specific shRNA (NON-SP shRNA). The normalized fluorescence intensity ratio of target (MyoD-EGFP) to internal control (RFP) confirmed the effectiveness of shRNA 708.

To demonstrate the ability of the MyoD-specific shRNA to inhibit expression of both ectopically expressed MyoD-EGFP and endogenous MyoD, cells were transfected with plasmid-encoded MyoD-specific shRNAs or a non-specific shRNA (NON-SP shRNA) and subjected to western blot analysis using an α-MyoD antibody (FIG. 5C). The results of this analysis showed a strong correlation between suppression of ectopic MyoD-EGFP and endogenous MyoD gene expression by the same panel of shRNAs, as MyoD-specific shRNA 708 again the most significant effect to suppress endogenous MyoD.

Lamin A/C. Five siRNAs targeting various regions spanning the Lamin A/C coding sequence were synthesized as described in Example 1 (see Table 1). These siRNAs were designated with respect to the transcription start site (nucleotide position 1) of Lamin A/C.

Human HeLa cells were co-transfected with EGFP-lamin A/C (prepared as described in Example 2), dSRed2-N1 (internal control for transfection), and Lamin A/C-specific siRNAs or non-specific siRNA (NON-SP). A siRNA (siRNA 608) known to be effective in mediating RNAi suppression of Lamin A/C expression (Harborth, et al., J Cell Sci 114, 4557-4565 (2001)) was included in the screen. 24 hours post-transfection, fluorescence microscopy images of EGFP and RFP were captured. Of the five siRNAs tested, siRNA 608 was by far the most effective in suppressing GFP reporter gene expression from the Lamin-GFP fusion.

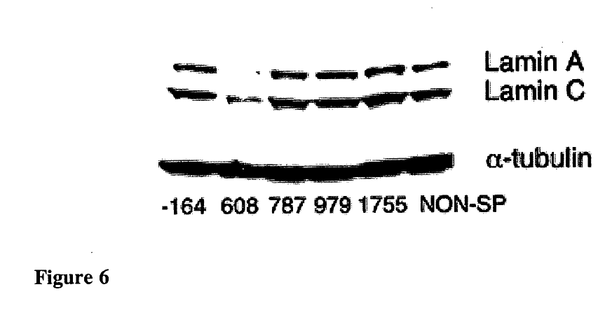

To demonstrate the correlation between the ability of the screened shRNAs to inhibit expression of both ectopically expressed Lamin A/C-EGFP and endogenous Lamin A/C, cells were transfected with siRNAs specific to Lamin A/C and subjected to western blot analysis using an a-Lamin A/C antibody (FIG. 6). The results of this analysis showed a strong correlation between suppression of ectopic Lamin A/C-EGFP and endogenous Lamin A and C expression by siRNAs, as siRNA 608 was the most effective in suppressing endogenous Lamin A and C expression.

Additional genes. We have screened a panel of siRNAs and shRNA probes against genes with diverse biological functions in both murine and human cell lines. Table 4 summarizes the screening results obtained with genes encoding murine helix-loop-helix gene transcription factor family members (Id1 through Id5), human tumor suppressor p53, and human EF-hand calcium binding protein S-100 α-subunit. For example, when a panel of shRNA probes against human tumor suppressor, p53 was examined, a published sequence (Brummelkamp, et al. Science 2002;296, 550-553) performed most efficiently of the 4 shRNAs tested in our screen (Table 2).

TABLE 4
siRNA/shRNA induced gene silencing for ectopically
expressed target-reporter fusions

| Gene | Genbank Accession # | siRNA* | shRNA* |
|---|---|---|---|
| MyoD | M84918 | 5(1) | 5(1) |
| Lamin A/C | NP_005563 | 5(1) | ND |
| S-100 | NM_002961.2 | 5(0) | ND |
| Id1 | AK008264 | 5(1) | 8(1) |
| Id2 | AF077860 | 5(0) | 3(1) |
| Id3 | AK002820 | 5(1) | 8(1) |
| Id4 | AF077859 | 5(1) | 3(0) |
| p53 | X02469 | 5(1) | 4(1) |

*Given as: number of RNAi probes tested(number of highly efficient RNAi probes).
ND = not done.

### Discussion

These results validate the reliability of this screening method to identify RNAi probes that efficiently suppress endogenous target gene expression. Furthermore, these results underscore that only a minority of RNAi probes are effective in gene silencing. This minority of RNAi probes can be rapidly and easily identified using this screening method. These data establish the correlation between the ability of an RNAi probe identified by the novel screening method with the ability of the identified RNAi probe to effectively suppress expression of the cognate endogenous gene.

A major strength of this method is its ability to identify the most robust siRNA candidate within 24 hours of transfection irrespective of the status of the endogenous protein. This is particularly attractive when compared to determining efficacy of siRNA probes by monitoring their ability to directly suppress cognate endogenous genes, which may involve time-consuming optimization with siRNA dose and incubation time (Elbashir, et al. Nature 2001;411, 494-498; Harborth, et al. J Cell Sci 2001;1 14, 4557-4565; Mendez, et al. Mol Cell. 2002;9, 481-91).

In addition to identifying the most effective siRNAs, we observed that other RNAi probes in the panel showed partial suppression of target gene expression. These RNAi probes would be useful in studies where partial down regulation of gene expression results in a discrete phenotype. For example, shRNAs showing varying levels of p53 suppression generated distinct tumor phenotypes in vivo (Hemann, et al. Nat Genet. 2003;33:396-400). These candidates may also be useful where lethality associated with complete suppression of critical genes is of concern.

### Example 5

### In Vivo High Throughput Selection of RNAi Probes

To demonstrate that this method for selecting effective siRNA probes would work in a highly parallel assay, we used a microarray based cell transfection method. Cell microarrays were printed, transfected, and processed essentially as described in Ziauddin and Sabatini, Nature 2001 ;411:107-110 and U.S. Application Publication No. 2002/000664. For complete experimental details concerning this method see U.S. Application Publication No. 2002/000664, hereby incorporated by reference. The protocol used is summarized below.

### Materials and Methods

Microarray printing. A robotic arrayer (VP478A, V & P Scientific, Inc. CA) was used to print a target gene-report fusion expression construct/RNAi probe/gelatin solution onto CMT GAPS glass slides (Corning, Inc.) at 4° C. The arrayer deposited about 1 nl volumes 400 μm apart using a 25-50-ms pin-down-slide time in a 55% relative humidity environment. Printed slides can be stored at 4° C. or at room temperature in a vacuum dessicator.

Preparation of aqueous gelatin solution is important and is as follows: 0.2% gelatin (w/v) (G-9391; Sigma) was dissolved in MilliQ water by heating and gentle swirling in a 60° C. water bath for 15 min. The solution was cooled slowly to room temperature and filtered through a 0.45-μm cellular acetate membrane and stored at 4° C. The deposited expression construct/RNAi probe/gelatin solution contained a final gelatin concentration of greater than 0.17%.

In the deposited solution, the final concentrations for EGFP fusion construct or pEGFP-N2 and pdSRed2-N1 (internal control) were 150 ng/μl and 50 ng/μl respectively. shRNA or siRNA concentration was kept constant at 300 ng/μl, or as mentioned.

Reverse transfection of microarrays. For transfections, 24 μl of Lipofectamine 2000 (Invitrogen) was mixed with 300 μl of OPTI-MEM I media (GibcoBRL) and pipetted onto a 40×20×0.2 mm cover well (PC200; Grace Bio-Labs). A microarray printed slide was placed printed side down on the cover well, such that the solution covered the entire arrayed area and created an airtight seal. After a 45 min incubation, the cover well was removed from the slide with forceps and the transfection reagent removed carefully by vacuum aspiration. The printed slide was then placed printed side up in a tissue culture dish, and incubated with 1×10⁶ HeLa cells per ml of culture medium (DMEM supplemented with 10% FBS, 100 U/ml penicillin and 100 μg/ml streptomycin (Life Technologies, Rockville, Md.)) at 37° C. in a 5% CO₂-humidified chamber. The HeLa cells were cultured on the printed slide for 24 hours with a media change at 6 hours. The cells on the slide were then fixed for 20 min at room temperature in 3.7% paraformaldehyde/4.0% sucrose in PBS, and mounted with a coverslip.

Laser scanning and fluorescence microscopy. The slides were scanned using a laser scanner (ScanArray 5000; PerkinElmer) at 20 μM resolution to measure EGFP and RFP fluorescence. To obtain images at cellular resolution, cells were photographed with a conventional fluorescence microscope. Post scanning, the EGFP and RFP intensities of each spot were quantitated by GenePix 4.0 software (Axon Instruments, Foster City, Calif.). In all analysis, features showing obvious blemishes and morphological defects were eliminated as a control for cell viability.

Normalized mean intensities of fluorescence (EGFP/RFP). Normalized mean intensities of fluorescence (EGFP/RFP) were then calculated based on GenePix 4.0 software quantitations. The EGFP/RFP ratio measures EGFP fluorescence of a transfected cell cluster relative to RFP fluorescence of the cell cluster (as a control for transfection efficiency) at a given concentration of co-transfected siRNA. Each spot was represented in quadruplet and mean values were used for final quantitation. Features with low intensities (<100 units) in the red channel (RFP fluorescence) were considered to be inefficient transfections and removed from further analysis. Data used to calculate mean values was normalized to reduce the effects of outliers by exclusion of the highest 5% of the values and the lowest 5% of the values from the calculated mean.

### Results

In a first assay, the microarray was used to transfect HeLa cells with pEGFP-N2 as the target gene expression construct, the RFP expression construct pDsRed2-N1 as an internal control, and varying concentrations of either EGFP-specific (EGFP-SP) or non-specific (NON-SP) siRNAs (see Example 1 and Table 1, above).

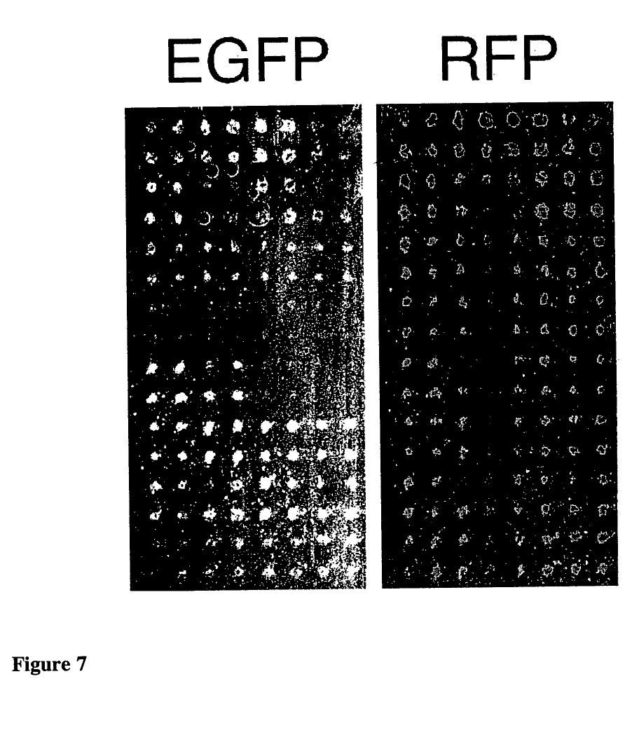

Using this method, only the cells growing in close proximity to the printed target gene-report fusion expression construct/RNAi probe/gelatin spots were transfected, driving expression of fusion proteins in spatially distinct groups of cells within a lawn of untransfected cells. A laser scanner was used to monitor fluorescence intensity changes in each individual transfected cell cluster. Laser scan fluorescence images showing microarray cell clusters expressing EGFP and RFP are shown (FIG. 7). Each cell cluster was ˜500 μM in diameter with a pitch of 750 μM. Typically each cluster was comprised of 300-500 fluorescent cells.

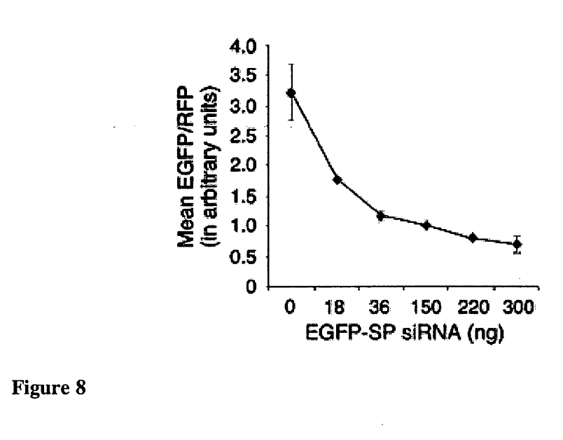

Normalized mean intensities of fluorescence (EGFP/RFP) were then quantitated. The mean EGFP/RFP values for cell clusters transfected with a given concentration of co-transfected EGFP-SP siRNA (♦) were plotted versus increasing concentration of co-transfected siRNA (FIG. 8). This graph reveals dose dependent suppression of. EGFP expression by its specific siRNA (EGFP-SP), with 300 ng of siRNA providing maximal suppression. This result established that the microarray format recapitulates the siRNA-mediated suppression of ectopic gene expression as a function of siRNA concentration observed previously in conventional transfections (see FIG. 4).

The use of such cell microarrays in screens to identify effective RNAi probes was then verified in a second assay. The microarray technique was used to transfect cells with the MyoD-EGFP expression construct (see Example 2D), the pDsRed-N1 RFP expression construct as an internal control, and a panel of 6 siRNAs and 6 shRNAs for MyoD (5 MyoD-specific and 1 non-specific shRNA or siRNA control (NON-SP) in each panel (See Table 1 and Table 3). These RNAi probes were analyzed for their ability to suppress expression of ectopic MyoD-EGFP, with RFP as an internal control.

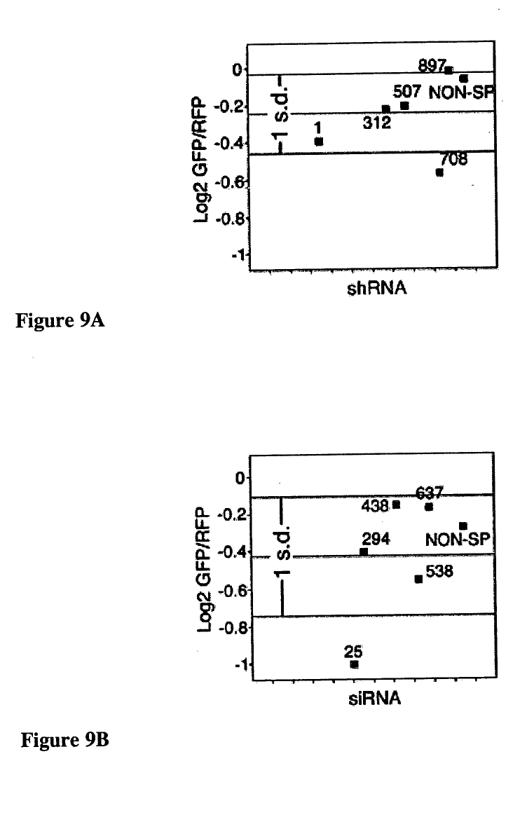

Mean intensities of fluorescence (EGFP/RFP) were log transformed, normalized (n=4), and plotted in a graph on the Y-axis versus individual siRNA/shRNA probes on the X-axis (FIG. 9A for shRNAs and FIG. 9B for siRNAs). In each case probes within 1 standard deviation from the mean value were considered non-effective; and those outside 1 standard deviation was considered effective. This analysis identified shRNA 708 and siRNA 25 as the most effective RNAi probes for suppression of MyoD-EGFP expression, a result in agreement with those from conventional transfections (see FIG. 5).

These results established that microarray techniques can be used for large scale screens to identify effective RNAi probes. For example, using fully automated liquid-dispensing and plate handling robotic systems it is possible to assemble constructs expressing target-reporter fusions, internal controls, various shRNAs and siRNAs that can be printed at densities of up to 6,000 to 10,000 features per slide by modern microarrayers.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Numerous references, including patents, patent applications, and various publications are cited and discussed in the description of this invention. The citation and/or discussion of such references is provided merely to clarify the description of the present invention and is not an admission that any such reference is “prior art” to the present invention. All references cited and discussed in this specification are incorporated herein by reference in their entirety and to the same extent as if each reference was individually incorporated by reference.

1. A method of determining whether an RNAi probe can inhibit expression of a target gene, which method comprises detecting expression of (i) a target-reporter fusion construct in a first cell transfected with a candidate RNAi molecule and the target-reporter fusion construct, wherein the target-reporter fusion construct comprises a reporter gene fused to the target nucleic acid, and (ii) the target-reporter fusion construct in a second cell transfected with the target-reporter fusion construct, wherein the candidate RNAi molecule inhibits expression of the target nucleic acid if the level of target-reporter fusion expression in the first cell is decreased as compared to the level of expression in the second cell.

2. The method according to claim 1, wherein the reporter is a fluorescent reporter.

3. The method according to claim 1, wherein the reporter is an enzymatic reporter.

4. The method according to claim 1, wherein the target-reporter fusion construct comprises a reporter gene-encoding sequence fused to the 5′ end of the target nucleic acid sequence.

5. The method according to claim 1, wherein the target-reporter fusion construct comprises a reporter gene-encoding sequence fused to the 3′ end of the target nucleic acid sequence.

6. The method according to claim 1, wherein the first and second cells are mammalian cells.

7. The method according to claim 1, wherein the first and second cells are also transfected with a second reporter gene, wherein the second reporter gene is different than the reporter gene in the target-reporter fusion construct and the second reporter gene serves as an internal control.

8. The method according to claim 2, wherein the detecting is done by measuring fluorescence intensity.

9. The method according to claim 8, wherein the detecting is done by laser scanning.

10. The method according to claim 8, wherein the fluorescence intensity is quantitated.

11. The method according to claim 1, wherein the detecting is done by immunoassay.

12. The method according to claim 11, wherein the immunoassay is western blot analysis or enzyme linked immunosorbent assay (ELISA).

13. The method according to claim 1, wherein the second cell is transfected with a non-specific RNAi molecule as a control.

14. A method of screening for candidate RNAi molecules that inhibit expression of a target nucleic acid, which method comprises
(a) arraying candidate RNAi molecules and a target-reporter fusion construct onto a surface, wherein the target-reporter fusion construct comprises a reporter gene fused to the target nucleic acid, and each candidate RNAi molecule is localized to a spatially distinct spot on the surface;
(b) incubating the arrayed surface with cells under appropriate conditions for entry of nucleic acid molecules, wherein this incubation results in clusters of transfected cells; and
(c) detecting expression of the target-reporter fusion in the clusters of transfected cells,
wherein a candidate RNAi molecule inhibits expression of the target nucleic acid if the level of target-reporter fusion expression in the cluster of cells into which the candidate RNAi molecule was transfected is decreased as compared to the level of expression in other clusters of cells.

15. The method according to claim 14, wherein a protein carrier is also arrayed onto the surface.

16. The method according to claim 15, wherein a protein carrier is gelatin.

17. The method according to claim 14, wherein the surface is a glass slide.

18. The method according to claim 14, wherein the arrayed surface is incubated with a transfection reagent and culture medium.

19. The method according to claim 14, wherein the reporter is a fluorescent reporter.

20. The method according to claim 19, wherein the detecting is done by measuring fluorescence intensity.

21. The method according to claim 14, wherein the cells are also transfected with a second reporter gene, wherein the second reporter gene is different than the reporter gene in the target-reporter fusion construct and the second reporter gene serves as an internal control.

22. A method of screening for candidate RNAi molecules that inhibit expression of a target nucleic acid, which method comprises
(a) depositing a nucleic acid-containing mixture onto a surface in discrete, defined locations, wherein the nucleic acid-containing mixture comprises a target-reporter fusion construct comprising a reporter gene fused to the target nucleic acid, a candidate RNAi molecule, and a carrier protein and allowing the nucleic acid-containing mixture to dry on the surface, thereby producing a surface having the nucleic acid-containing mixture affixed thereon in discrete, defined locations,
(b) plating eukaryotic cells onto the surface in sufficient density and under appropriate conditions for entry of nucleic acid in the nucleic acid-containing mixture into the eukaryotic cells, whereby nucleic acid in the nucleic acid-containing mixture is introduced into the eukaryotic cells, resulting in clusters of transfected cells; and
(c) detecting expression of the target-reporter fusion in the clusters of transfected cells,
wherein a candidate RNAi molecule inhibits expression of the target nucleic acid if the level of target-reporter fusion expression in the cluster of cells into which the RNAi probe was transfected is decreased as compared to the level of expression in other clusters of transfected cells.

23. The method according to claim 22, wherein a protein carrier is also arrayed onto the surface.

24. The method according to claim 23, wherein a protein carrier is gelatin.

25. The method according to claim 22, wherein the surface is a glass slide.


SEQUENCE LISTING (66 sequences)

SEQ ID NO: 1 - DNA, 506 bp, Homo sapiens
  misc_feature (1)..(506): where n may be a or g or c ot t/u or unknown
aagcttggctgcaggtcgacggatccccccgagtccaacacccgtgggaatcccatggnnaccatggcccctcgctccaaaaatgctttcgcgtctcgcagacactgctcggtagtttcggggatcagcgtttgagtaagagcccgcgtctgaaccctccgcgccgccccggncccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagtttttaaaattatgttttaaaatggactatcatatgcttaccgtaacttgaaagtatttcgatttcttggctttatatatcttgtggaaaggacgaaacacc

SEQ ID NO: 2 - DNA, 18 bp, Artificial Sequence
  desalted DNA oligonucleotides
taatacgactcactatag

SEQ ID NO: 3 - DNA, 39 bp, Artificial Sequence
  desalted DNA oligonucleotide
atgaacttcagggtcagcttgctatagtgagtcgtatta

SEQ ID NO: 4 - DNA, 39 bp, Artificial Sequence
  desalted DNA oligonucleotide
cggcaagctgaccctgaagttctatagtgagtcgtatta

SEQ ID NO: 5 - DNA, 39 bp, Artificial Sequence
  desalted DNA oligonucleotide
atgatactcgagggcatgtctctatagtgagtcgtatta

SEQ ID NO: 6 - DNA, 39 bp, Artificial Sequence
  desalted DNA oligonucleotide
cggagacatgccctcgagtatctatagtgagtcgtatta

SEQ ID NO: 7 - DNA, 22 bp, Artificial Sequence
  EGFP-specific siRNA probe
gcaagcugacccugaaguucau

SEQ ID NO: 8 - DNA, 22 bp, Artificial Sequence
  EGFP-specific siRNA probe
gaacuucagggucagcuugccg

SEQ ID NO: 9 - DNA, 22 bp, Artificial Sequence
  non-specific siRNA probe
gagacaugcccucgaguaucau

SEQ ID NO: 10 - DNA, 22 bp, Artificial Sequence
  non-specific siRNA probe
gauacucgagggcaugucuccg

SEQ ID NO: 11 - DNA, 21 bp, Artificial Sequence
  MyoD-specific 25 siRNA probe
ggccugucaagucuauguccc

SEQ ID NO: 12 - DNA, 21 bp, Artificial Sequence
  MyoD-specific 25 siRNA probe
ggcauagacuugacaggcccc

SEQ ID NO: 13 - DNA, 21 bp, Artificial Sequence
  MyoD-specific 294 siRNA probe
ggucuugcgcuugcacgccuu

SEQ ID NO: 14 - DNA, 21 bp, Artificial Sequence
  MyoD-specific 294 siRNA probe
ggcgugcaagcgcaagaccac

SEQ ID NO: 15 - DNA, 21 bp, Artificial Sequence
  MyoD-specific 438 siRNA probe
ggcguugcgcaggaucuccac

SEQ ID NO: 16 - DNA, 21 bp, Artificial Sequence
  MyoD-specific 438 siRNA probe
ggagauccugcgcaacgccau

SEQ ID NO: 17 - DNA, 21 bp, Artificial Sequence
  MyoD-specific 538 siRNA probe
ggccugggggcagcgguccag

SEQ ID NO: 18 - DNA, 21 bp, Artificial Sequence
  MyoD-specific 538 siRNA probe
ggaccgcugcccccaggccgu

SEQ ID NO: 19 - DNA, 21 bp, Artificial Sequence
  MyoD-specific 637 siRNA probe
gggggccgcuuggggggccgc

SEQ ID NO: 20 - DNA, 21 bp, Artificial Sequence
  MyoD-specific 637 siRNA probe
ggccccccaagcggcccccgg

SEQ ID NO: 21 - DNA, 21 bp, Artificial Sequence
  Lamin A/C-specific -164 siRNA probe
ggccgggcgcugucggaccuc

SEQ ID NO: 22 - DNA, 21 bp, Artificial Sequence
  Lamin A/C-specific -164 siRNA probe
gguccgacagcgcccggccca

SEQ ID NO: 23 - DNA, 23 bp, Artificial Sequence
  Lamin A/C-specific 608 siRNA probe
cuggacuuccagaagaacauctt

SEQ ID NO: 24 - DNA, 23 bp, Artificial Sequence
  Lamin A/C-specific 608 siRNA probe
gauguucuucuggaaguccagtt

SEQ ID NO: 25 - DNA, 21 bp, Artificial Sequence
  Lamin A/C-specific 787 siRNA probe
ggcagaauaagucuucuccag

SEQ ID NO: 26 - DNA, 21 bp, Artificial Sequence
  Lamin A/C-specific 787 siRNA probe
ggagaagacuuauucugccaa

SEQ ID NO: 27 - DNA, 21 bp, Artificial Sequence
  Lamin A/C-specific 979 siRNA probe
ggugucccgcucacgggccag

SEQ ID NO: 28 - DNA, 21 bp, Artificial Sequence
  Lamin A/C-specific 979 siRNA probe
ggcccgugagcgggacaccag

SEQ ID NO: 29 - DNA, 21 bp, Artificial Sequence
  Lamin A/C-specific 1755 siRNA probe
ggcuggggagaggcugccccc

SEQ ID NO: 30 - DNA, 21 bp, Artificial Sequence
  Lamin A/C-specific 1755 siRNA probe
gggcagccucuccccagccuc

SEQ ID NO: 31 - DNA, 39 bp, Artificial Sequence
  MyoD 25 desalted DNA oligonucleotide primer
gggacatagacttgacaggcctatagtgagtcgtattac

SEQ ID NO: 32 - DNA, 39 bp, Artificial Sequence
  MyoD 25 desalted DNA oligonucleotide primer
ggggcctgtcaagtctatgcctatagtgagtcgtattac

SEQ ID NO: 33 - DNA, 39 bp, Artificial Sequence
  MyoD 294 desalted DNA oligonucleotide primer
aaggcgtgcaagcgcaagacctatagtgagtcgtattac

SEQ ID NO: 34 - DNA, 39 bp, Artificial Sequence
  MyoD 294 desalted DNA olignucleotide
gtggtcttgcgcttgcacgcctatagtgagtcgtattac

SEQ ID NO: 35 - DNA, 39 bp, Artificial Sequence
  MyoD 438 desalted DNA oligonucleotide primer
gtggagatcctgcgcaacgcctatagtgagtcgtattac

SEQ ID NO: 36 - DNA, 39 bp, Artificial Sequence
  MyoD 438 desalted DNA oligonucleotide primer
atggcgttgcgcaggatctcctatagtgagtcgtattac

SEQ ID NO: 37 - DNA, 39 bp, Artificial Sequence
  MyoD 538 desalted DNA oligonucleotide primer
ctggaccgctgcccccaggcctatagtgagtcgtattac

SEQ ID NO: 38 - DNA, 39 bp, Artificial Sequence
  MyoD 538 desalted DNA oligonucleotide primer
acggcctgggggcagcggtcctatagtgagtcgtattac

SEQ ID NO: 39 - DNA, 39 bp, Artificial Sequence
  MyoD 637 desalted DNA oligonucleotide primer
gcggccccccaagcggccccctatagtgagtcgtattac

SEQ ID NO: 40 - DNA, 39 bp, Artificial Sequence
  MyoD 637 desalted DNA oligonucleotide primer
ccgggggccgcttggggggcctatagtgagtcgtattac

SEQ ID NO: 41 - DNA, 39 bp, Artificial Sequence
  Lamin A/C -164 desalted DNA oligonucleotide primer
gaggtccgacagcgcccggcctatagtgagtcgtattac

SEQ ID NO: 42 - DNA, 39 bp, Artificial Sequence
  Lamin A/C -164 desalted DNA oligonucleotide primer
tgggccgggcgctgtcggacctatagtgagtcgtattac

SEQ ID NO: 43 - DNA, 39 bp, Artificial Sequence
  Lamin A/C 787 desalted DNA oligonucleotide primer
ctggagaagacttattctgcctatagtgagtcgtattac

SEQ ID NO: 44 - DNA, 39 bp, Artificial Sequence
  Lamin A/C 787 desalted DNA oligonucleotide
ttggcagaataagtcttctcctatagtgagtcgtattac

SEQ ID NO: 45 - DNA, 39 bp, Artificial Sequence
  Lamin A/C 979 desalted DNA oligonucleotide primer
ctggcccgtgagcgggacacctatagtgagtcgtattac

SEQ ID NO: 46 - DNA, 39 bp, Artificial Sequence
  Lamin A/C 979 desalted DNA oligonucleotide primer
ctggtgtcccgctcacgggcctatagtgagtcgtattac

SEQ ID NO: 47 - DNA, 39 bp, Artificial Sequence
  Lamin A/C 1755 desalted DNA oligonucleotide primer
gggggcagcctctccccagcctatagtgagtcgtattac

SEQ ID NO: 48 - DNA, 39 bp, Artificial Sequence
  Lamin A/C 1755 desalted DNA oligonucleotide primer
gaggctggggagaggctgccctatagtgagtcgtattac

SEQ ID NO: 49 - DNA, 71 bp, Artificial Sequence
  non-specific shRNA
tccaattcagcgggagccacctgatgaagcttgatcgggtggctctcgctgagttggaatccatttttttt

SEQ ID NO: 50 - DNA, 71 bp, Artificial Sequence
  non-specific shRNA
aaaaaaaatggattccaactcagcgagagccacccgatcaagcttcatcaggtggctcccgctgaattgga

SEQ ID NO: 51 - DNA, 73 bp, Artificial Sequence
  MyoD-specific 1 shRNA
tcccggagtggcggcgatagaagctccagaagcttgtggagcttctgtcgccgccgcttcgggatattttttt

SEQ ID NO: 52 - DNA, 79 bp, Artificial Sequence
  MyoD-specific 1 shRNA
gatcaaaaaaatatcccgaagcggcggcgacagaagctccacaagcttctggagcttctatcgccgccactccgggacg

SEQ ID NO: 53 - DNA, 73 bp, Artificial Sequence
  MyoD-specific 312 shRNA
cggccttgcggcgatcagcgttggtggtgaagcttgatcaccagcgctggtcgccgcaaggtcgccatttttt

SEQ ID NO: 54 - DNA, 79 bp, Artificial Sequence
  MyoD-specific 312 shRNA
gatcaaaaaatggcgaccttgcggcgaccagcgctggtgatcaagcttcaccaccaacgctgatcgccgcaaggccgcg

SEQ ID NO: 55 - DNA, 73 bp, Artificial Sequence
  MyoD-specific 507 shRNA
cgtagaaggcagcggcgccagggggcgcgaagcttggtgccccttggcgtcgctgtcttctacgcactttttt

SEQ ID NO: 56 - DNA, 79 bp, Artificial Sequence
  MyoD-specific 507 shRNA
gatcaaaaaagtgcgtagaagacagcgacgccaaggggcaccaagcttcgcgccccctggcgccgctgccttctacgcg

SEQ ID NO: 57 - DNA, 73 bp, Artificial Sequence
  MyoD-specific 708 shRNA
acacagccgcactcttccctggcctggagaagcttgttcaggctagggaggagtgtggctgtgtcgatttttt

SEQ ID NO: 58 - DNA, 79 bp, Artificial Sequence
  MyoD-specific 708 shRNA
gatcaaaaaatcgacacagccacactcctccctagccygaacaagcttctccaggccagggaagagtgcggctgtgtcg

SEQ ID NO: 59 - DNA, 73 bp, Artificial Sequence
  MyoD-specific 897 shRNA
agcctgcaggacactgaggggcggcgtcgaagcttgggcgccgtccctcggtgtcttgcaggctcaatttttt

SEQ ID NO: 60 - DNA, 79 bp, Artificial Sequence
  MyoD-specific 897 shRNA
gatcaaaaaattgagcctgcaagacaccgagggacggcgcccaagcttcgacgccgcccctcagtgtcctgcaggctcg

SEQ ID NO: 61 - DNA, 30 bp, Artificial Sequence
  PCR primer RFP-1
tttttggatcccatacaggaacaggtggtg

SEQ ID NO: 62 - DNA, 26 bp, Artificial Sequence
  PCR primer RFP-2
cgccagcaacaacgcggcctttttac

SEQ ID NO: 63 - DNA, 30 bp, Artificial Sequence
  PCR primer EGFP-1
ttttggatcccgatacttgtacagctcgtc

SEQ ID NO: 64 - DNA, 26 bp, Artificial Sequence
  PCR primer EGFP-2
cgccagcaacaacgcggcctttttac

SEQ ID NO: 65 - DNA, 28 bp, Artificial Sequence
  PCR primer MyoD-1
ttttctcgagatggagcttctatcgccg

SEQ ID NO: 66 - DNA, 27 bp, Artificial Sequence
  PCR primer MyoD-2
gtggatcccacaaagcacctgataaat